(19) United States
(12) Patent Application Publication
(10) Pub. No.: US 2003/0109683 A1
(43) Pub. Date: Jun. 12, 2003

(54) Mutated steroid hormone receptors, methods for their use and molecular switch for gene therapy

(75) Inventors: Bert W. O'Malley, Houston, TX (US); Ming-Jer Tsai, Houston, TX (US); Sophia Y. Tsai, Houston, TX (US); Harry C. Ledebur JR., Spring, TX (US); Yaolin Wang, Iselin, NJ (US); Joseph D. Kittle JR., Houston, TX (US)

Correspondence Address: PERKINS COIE LLP, WO, POST OFFICE BOX 1208, SEATTLE, WA, 98111-1208, US

(73) Assignees: Baylor College of Medicine; Valentis, Inc.

(21) Appl. No.: 10/124,741

(22) Filed: Apr. 16, 2002

Related U.S. Application Data
(63) Continuation of application No. 09/916,145, filed on Jul. 25, 2001, now abandoned.
Application No. 09/916,145 is a continuation of application No. 08/959,013, filed on Oct. 28, 1997, now abandoned.
(63) Continuation-in-part of application No. 08/479,913, filed on Jun. 7, 1995, now Pat. No. 6,416,998.
(63) Continuation-in-part of application No. 09/465,133, filed on Dec. 15, 1999.
Application No. 09/465,133 is a continuation of application No. 09/209,981, filed on Dec. 9, 1998.
Application No. 09/209,981 is a division of application No. 08/479,846, filed on Jun. 6, 1995, now Pat. No. 5,874,534.
Application No. 08/479,846 is a continuation of application No. 07/939,246, filed on Sep. 2, 1992, now abandoned.
Application No. 07/939,246 is a continuation-in-part of application No. 07/882,771, filed on May 14, 1992, now Pat. No. 5,364,791.
(60) Provisional application No. 60/029,964, filed on Oct. 29, 1996.

(30) Foreign Application Priority Data
Oct. 28, 1997 (WO) PCT/US97/19607

(51) Int. Cl. C07K 14/72; C07H 21/04; C12P 21/02; C12N 5/06

(52) U.S. Cl. 530/395; 536/23.5; 435/69.7; 435/320.1; 435/325

(57) ABSTRACT

The present invention provides mutant proteins of steroid hormone receptors. These mutant proteins are useful in methods of distinguishing a steroid hormone receptor antagonist from a steroid hormone receptor agonist. The present invention also provides plasmids containing mutated steroid hormone receptor proteins and cells transfected with those plasmids. In addition, the present invention provides methods for determining whether a compound is a steroid hormone receptor antagonist or agonist. Also, the present invention provides methods of determining endogenous ligands for steroid hormone receptors. The invention further provides a molecular switch protein for regulating expression in gene therapy.

### CLAIM OF PRIORITY

The present application is a continuation of co-pending U.S. patent application Ser. No. 09/916,145, filed Jul. 25, 2001 entitled MODIFIED HORMONES FOR GENE THERAPY AND METHODS OF THEIR USE by O'Malley et al. (Lyon & Lyon Docket No. 264/035), which is a continuation of U.S. patent application Ser. No. 08/959,013, filed Oct. 28, 1997, now abandoned, entitled “MODIFIED STEROID HORMONES FOR GENE THERAPY AND METHODS OF THEIR USE” by O'Malley et al. (Lyon & Lyon Docket No. 226/286), which claims priority to U.S. Provisional Patent Application Serial No. 60/029,964, filed Oct. 29, 1996, entitled “MODIFIED STEROID HORMONES FOR GENE THERAPY AND METHODS OF THEIR USE” by O'Malley et al. (Lyon & Lyon Docket No. 222/085);

The present application is a continuation-in-part of co-pending U.S. application Ser. No. 08/479, 913 (Lyon & Lyon Docket No. 212/133), O'Malley et al., filed Jun. 7,1995, entitled “MODIFIED STEROID HORMONES FOR GENE THERAPY AND METHODS OF THEIR USE.”

The present application is a continuation-in-part of co-pending U.S. application Ser. No. 09/465,133 (Lyon & Lyon Docket No. 246/180), Vegeto et al., filed Dec. 16, 1999, entitled “MUTATED STEROID HORMONE RECEPTORS, METHODS FOR THEIR USE AND MOLECULAR SWITCH FOR GENE THERAPY,” which is a continuation application of U.S. application Ser. No. 09/209,981 (Lyon & Lyon Docket No. 237/177), Vegeto et al., filed Dec. 9, 1998, entitled “MUTATED STEROID HORMONE RECEPTORS, METHODS FOR THEIR USE AND MOLECULAR SWITCH FOR GENE THERAPY,” which is a divisional application of U.S. application Ser. No. 08/479,846, filed Jun. 6, 1995, now U.S. Pat. No. 5,874,534, Vegeto et al, issued Feb. 23, 1999, entitled “MUTATED STEROID HORMONE RECEPTORS, METHODS FOR THEIR USE AND MOLECULAR SWITCH FOR GENE THERAPY,” which is a continuation of U.S. application Ser. No. 07/939,246, Vegeto et al., filed Sep. 2, 1992, entitled “MUTATED STEROID HORMONE RECEPTORS, METHODS FOR THEIR USE AND MOLECULAR SWITCH FOR GENE THERAPY,” now abandoned, which is a continuation-in-part of U.S. application Ser. No. 07/882,771, filed May 14, 1992, now U.S. Pat. No. 5,364,791, entitled “PROGESTERONE RECEPTOR HAVING C TERMINAL HORMONE BINDING DOMAIN TRUNCATIONS.”

The invention described herein was developed in part with funds provided by the National Institutes of Health, Grant Number HD07857. The Government has certain rights.

The present application claims priority to the filing dates of each of the above-identified applications which are hereby incorporated by reference (including drawings) as if fully set forth herein.

### FIELD OF THE INVENTION

The present invention relates generally to the fields of molecular endocrinology and receptor pharmacology. It further relates to molecular switches for gene therapy. More specifically, the present invention relates to a novel in vivo method for the identification of steroid hormone receptor agonists and antagonists and to a molecular switch involving a modified steroid receptor for up-regulating and down-regulating the synthesis of heterologous nucleic acid sequences which have been inserted into cells. This invention has applicability to gene therapy, whereby modified steroid receptors regulate the expression of genes within tissue. In particular, the modified steroid receptors contain a DNA binding domain, one or more transregulatory domains, and a ligand binding domain and are capable of binding a non-natural ligand.

### BACKGROUND OF THE INVENTION

Steroid receptors are responsible for the regulation of complex cellular events, including transcription. The ovarian hormones, estrogen and progesterone, are responsible, in part, for the regulation of the complex cellular events associated with differentiation, growth and functioning of female reproductive tissues. These hormones play also important roles in development and progression of malignancies of the reproductive endocrine system.

The biological activity of steroid hormones is mediated directly by a hormone and tissue-specific intracellular receptor. The physiologically inactive form of the receptor may exist as an oligomeric complex with proteins, such as heat-shock protein (hsp) 90, hsp70 and hsp56. Upon binding its cognate ligand, the receptor changes conformation and dissociates from the inhibitory heteroligomeric complex. Subsequent dimerization allows the receptor to bind to specific DNA sites in the regulatory region of target gene promoters. Following binding of the receptor to DNA, the hormone is responsible for mediating a second function that allows the receptor to interact specifically with the transcription apparatus. Displacement of additional inhibitory proteins and DNA-dependent phosphorylation may constitute the final steps in this activation pathway.

Cloning of several members of the steroid receptor superfamily has facilitated the reconstitution of hormone-dependent transcription in heterologous cell systems. Subsequently, in vivo and in vitro studies with mutant and chimeric receptors have demonstrated that steroid hormone receptors are modular proteins organized into structurally and functionally defined domains. A well-defined 66 amino acid DNA binding domain (DBD) has been identified and studied in detail, using both genetic and biochemical approaches. The ligand (hormone) binding domain (LBD), located in the carboxyl-terminal half of the receptor, consists of about 300 amino acids. It has not been amenable to detailed site-directed mutagenesis, since this domain appears to fold into a complex tertiary structure, creating a specific hydrophobic pocket which surrounds the effector molecule. This feature creates difficulty in distinguishing among amino acid residues that affect the overall structure of this domain from those involved in a. direct contact with the ligand. The LBD also contains sequences responsible for receptor dimerization, hsp interactions and one of the two transactivation sequences of the receptor.

Gene replacement therapy requires the ability to control the level of expression of transfected genes from outside the body. Such a “molecular switch” should allow specificity, selectivity, precision safety and rapid clearance. The steroid receptor family of gene regulatory proteins is an ideal set of such molecules. These proteins are ligand activated transcription factors whose ligands can range from steroids to retinoids, fatty acids, vitamins, thyroid hormones and other presently unidentified small molecules. These compounds bind to receptors and either up-regulate or down-regulate. The compounds are cleared from the body by existing mechanisms and the compounds are non-toxic.

The efficacy of a ligand is a consequence of its interaction with the receptor. This interaction can involve contacts causing the receptor to become active (agonist) or for the receptor to be inactive (antagonist). The affinity of antagonist activated receptors for DNA is similar to that of agonist-bound receptor. Nevertheless, in the presence of the antagonist, the receptor cannot activate transcription efficiently. Thus, both up and down regulation is possible by this pathway.

The present invention shows that receptors can be modified to allow them to bind various ligands whose structure differs dramatically from the naturally occurring ligands. Small C-terminal alternations in amino acid sequence, including truncation, result in altered affinity and altered function of the ligand. By screening receptor mutants, receptors can be customized to respond. to ligands which do not activate the host cells own receptors. Thus regulation of a desired transgene can be achieved using a ligand which will bind to and regulate a customized receptor.

Steroid receptors and other mammalian transcription regulators can function in yeast. This fact, coupled with the ease of genetic manipulation of yeast make it a useful system to study the mechanism of steroid hormone action.

The expression of most mammalian genes is intricately regulated in vivo in response to a wide range of stimuli, including physical (pressure, temperature, light), electrical (e.g. motor and sensory neuron signal transmission) as well as biochemical (ions, nucleotides, neurotransmitters, steroids and peptides) in nature. While the mechanism of transcriptional regulation of gene expression has been extensively studied (McKnight, Genes Dev. 10:367-381 (1996)), progress on achieving target gene regulation in mammalian cells, without interfering with endogenous gene expression, has been limited. Currently, most strategies for target gene activation or repression are performed in a constitutive manner. Such uncontrolled regulation of gene expression is not ideal physiologically, and can even be deleterious to cell growth and differentiation. In contrast, use of the yeast GAL4 DNA binding domain in this invention does not interfere with endogenous genes since that chimeric regulator will only recognize target gene constructs containing the GAL4 binding sequence.

Several inducible systems have been employed for controlling target gene expression. These inducible agents include heavy metal ions (Mayo et al., Cell 29:99108 (1982)), heat shock (Nover et al. CRC Press 167-220 (1991)), isopropyl -D-thiogalactoside (IPTG) (Baim et al. Proc. Natl. Acad. Sci. 88:5072-5076 (1981)), and steroid hormones such as estrogen (Braselmann et al. Proc. Natl. Acad. Sci. 90:1657-1661 (1993)) and glucocorticoids (Lee et al. Nature 294:228-232 (1981)). However, many of these inducers are either toxic to mammalian cells or interfere with endogenous gene expression (Figge et al. Cell 52:713-722 (1988)).

Utilizing a bacterial tetracycline-responsive operon element, Gossen et al. developed a model for controlling gene expression with a tetracycline-controlled transactivator (tTA and rtTA) (Gossen et al. Proc. Natl. Acad. Sci. 89:5547-5551 (1992); Gossen et al. Science 268:17661769 (1995)). No et al. recently reported a three-component system consisting of a chimeric GAL4-VP16 ecdysone receptor, its partner retinoid X receptor (RXR), and a target gene. They demonstrated its application in activating reporter gene expression in an ecdysone-dependent manner (No et al. Proc. Natl. Acad. Sci. 93:3346-3351 (1996). The invention described herein has advantages over the No and Gossen models, as the chimeric regulator recognizes only the target gene constructs and not endogenous genes, and the system is only activated in the presence of an exogenous compound, but not in the presence of any endogenous molecules.

A long felt need and desire in this art would be met by the development of methods to identify steroid hormone receptors agonists and antagonists. The development of such a method will facilitate the identification of novel therapeutic pharmaceuticals. Additionally, the present invention provides a novel approach to regulate transcription in gene therapy. By using modified steroid receptors and custom ligands, up-regulation and down-regulation of inserted nucleic acid sequences can be achieved.

### SUMMARY OF THE INVENTION

An object of the present invention is a modified steroid hormone receptor protein for distinguishing hormone antagonists and agonists.

An additional object of the present invention is a plasmid containing a modified hormone receptor.

A further object of the present invention are transfected cells containing modified hormone receptors.

Another object of the present invention is a transformed cell containing modified hormone receptors.

An additional object of the present invention is a method for determining agonist activity of a compound for steroid hormone receptors.

A further object of the present invention is a method for determining antagonist activity of a compound for steroid hormone receptors.

An object of the present invention is a method for determining endogenous ligands for steroid hormone receptors.

An object of the present invention is an endogenous ligand for a modified steroid receptor.

An object of the present invention is a molecular switch for regulated expression of a nucleic acid sequence in gene therapy.

An additional object of the present invention is a molecular switch which binds non-natural ligands, anti-hormones and non-native ligands.

A further object of the present invention is a molecular switch comprised of a modified steroid receptor.

An additional object of the present invention is a method for regulating expression of nucleic acid sequence in gene therapy.

A further object of the present invention is a modified progesterone receptor with a native binding domain replaced with GAL-4 DNA.

An additional object of the present invention is to add a more potent activation domain to the receptor.

Another object of the present invention is a method of treating senile dementia or Parkinson's disease.

Thus, in accomplishing the foregoing objects, there is provided in accordance with one aspect of the present invention a mutated steroid hormone receptor protein. This mutated steroid hormone receptor protein is capable of distinguishing a steroid hormone receptor antagonist from a steroid hormone receptor agonist.

In specific embodiments of the present invention, the receptor is selected from a group consisting of estrogen, progesterone, androgen, Vitamin D, COUP-TF, cis-retonic acid, Nurr-1, thyroid hormone, mineralocorticoid, glucocorticoid-α, glucocorticoid-β, ecdysterone and orphan receptors.

In a preferred embodiment the mutated steroid receptor is mutated by deletion of carboxy terminal amino acids. Deletion usually comprises from one to about 120 amino acids and is most preferably less than about 60 amino acids.

In another embodiment of the present invention, there is provided a plasmid containing a mutated steroid hormone receptor protein. The plasmid of the present invention when transfected into a cell, is useful in determining the relative antagonist or agonist activity of a compound for a steroid hormone receptor.

In another embodiment of the present invention, there is provided transfected and transformed cells containing a plasmid in which a mutated or steroid hormone receptor protein has been inserted. The transfected cells of the present invention are useful in methods of determining the activity of a compound for a steroid hormone receptor.

Another embodiment of the present invention, includes methods of determining whether a compound has activity as an agonist or antagonist as a steroid hormone receptor. These methods comprise contacting the compound of interest with the transfected cells of the present invention and measuring the transcription levels induced by the compound to determine the relative agonist or antagonist activity of the steroid hormone receptors.

In other embodiments of the present invention, there is provided a method of determining an endogenous ligand for a steroid hormone receptor. This method comprises contacting a compound with the transfected cells of the present invention and measuring the transcription levels induced by the compound.

Another embodiment of the present invention is the provision of endogenous ligands for modified steroid hormone receptors that are capable of stimulating transcription in the presence of the transfected cells of the present invention.

A further embodiment of the present invention is a molecular switch for regulating expression of a nucleic acid sequence in gene therapy in humans and animals. It is also useful as a molecular switch in plants and in transgenic animals. The molecular switch is comprised of a modified steroid receptor which includes a natural steroid receptor DNA binding domain attached to a modified ligand binding domain on said receptor.

In specific embodiments of the molecular switch, the native DNA binding domain in unmodified form is used and the ligand binding domain is modified to only bind a compound selected from the group consisting of non-natural ligands, anti-hormones and non-native ligands.

Specific examples of compounds which bind the ligand binding domain include 5-alpha-pregnane-3,20-dione; 11β-(4-dimethylaminophenyl)-17β-hydroxy-17α-ropinyl-4,9-estradiene-3-one; 11β-(4-dimethylaminophenyl)-17α-hydroxy-17β-(3-hydroxypropyl)-13α-methyl-4,9-gonadiene-3-one; 11β-(4-acetylphenyl)-17β-hydroxy-17α-(1-propinyl)-4,9-estradiene-3-one; 11β-(4-dimethylaminophenyl)-17β-hydroxy-17α-(3-hydroxy-1(Z)-propenyl-estra-4,9-diene-3-one; (7β, 11β, 17β)-11-(4-dimethylaminophenyl)-7-methyl-4′,5′-dihydrospiro[ester-4,9-diene-17,2′(3′H)-furan]-3-one; (11β,14β,17α)-4′,5′-dihydro-11-(4-dimethylaminophenyl)-[spiroestra-4,9-diene-17,2′(3′H)-furan]-3-one.

In preferred embodiments of the molecular switch, the modified steroid receptor has both the ligand binding domain and DNA binding domain replaced. For example the natural DNA binding domain is replaced with a DNA binding domain selected from the group consisting of GAL-4 DNA, virus DNA binding site, insect DNA binding site and a non-mammalian DNA binding site.

In specific embodiments of the present invention the molecular switch can further include transactivation domains selected from the group consisting of VP-16, TAF-1, TAF-2, TAU-1 and TAU-2.

In a preferred embodiment the molecular switch has a modified progesterone receptor containing a modified ligand binding domain and a GAL-4 DNA binding domain. This molecular switch can also be enhanced by the addition of a TAF-1 or VP16 transactivation domain.

Additional embodiments of the present invention include a method for regulating the expression of a nucleic acid cassette in gene therapy. The method includes the step of attaching the molecular switch to a nucleic acid cassette used in gene therapy. A sufficient dose of the nucleic acid cassette with the attached molecular switch is then introduced into an animal or human to be treated. The molecular switch can then be up-regulated or down-regulated by dosing the animal or human with a ligand which binds the modified binding site.

In another embodiment of the present invention, construction of novel modified steroid hormone receptors which regulate the expression of nucleic acid sequences is described herein, and surprisingly these modifications allow control of the transactivation and transrepressing functions of the modified steroid hormone receptor. Such modifications unexpectedly allow the receptors to bind various ligands whose structures differ dramatically from the naturally-occurring ligands (for example, non-natural ligands, anti-hormones and non-native ligands) and thereby provide a substantial improvement over prior attempts to control or regulate target gene expression.

These modified steroid receptors exhibit normal transactivation and transrepression activity; however, stimulation of such activity occurs via activation by a non-natural and exogenously or endogenously applied ligand. Modifications are also generated in the ligand binding domain of the PR and eliminate the ability of PR to bind its natural ligand.

Modifications allow the modified receptor to bind non-natural ligands and stimulate the transrepression of gene expression but not transactivation. Likewise, using the same ligand binding domain modification in conjunction with modifications to the transregulatory domain allows the modified receptor to bind non-natural ligands and stimulate transactivation but not transrepression of gene expression.

In one embodiment, the mutated ligand binding region is preferably mutated by deletion of about 16 to 42 carboxyl terminal amino acids of a progesterone receptor ligand binding domain. The mutated progesterone receptor ligand binding region preferably comprises, consists essentially of, or consists of about amino acids 640 through 891 of a progesterone receptor. Other preferred embodiments comprise, consist essentially of, or consist of amino acids 640-917, amino acids 640-920 or amino acids 640-914. One skilled in the art will recognize that various mutations can be created to achieve the desired function.

In one embodiment, the modified steroid hormone receptor protein includes a mutated progesterone ligand binding region of amino acids 640 through 914 of a progesterone receptor ligand binding domain. In another embodiment, the modified steroid hormone receptor protein contains a transregulatory domain located in the N-terminal region of the mutated progesterone receptor. In another embodiment, the modified steroid hormone receptor protein includes a transregulatory domain located in the C-terminal region of the mutated progesterone receptor. Thus, the transregulatory domain can be located either in the C-terminal or N-terminal direction of the mutated receptor.

In another embodiment, the modified steroid hormone receptor protein includes a GAL4 DNA binding domain. In another embodiment, the modified steroid hormone receptor protein includes a Krüppel-associated box-A (KRAB) transrepressing domain. The terms “GAL4 DNA binding domain” and “KRAB-transrepressing domain” are used as conventionally understood in the art and encompass functional equivalents of such sequences that retain the ability to bind DNA or retain the transrepressing activity.

In another embodiment, the modified steroid hormone receptor protein includes a mutated progesterone receptor ligand binding region capable of binding RU486 at a concentration as low as 0.01 nM. In still another embodiment, a modified steroid hormone receptor protein responds to a conventional antagonist of the wild-type steroid hormone receptor protein counterpart with an agonistic response. Those skilled in the art will understand that “binding” can be measured by several conventional methods in the art, such as binding constants and that a protein “response” can also be measured using conventional techniques in the art, such as measurement of induced transcription levels.

In a preferred embodiment, the modified steroid hormone receptor protein activates target gene expression. In another preferred embodiment, the target gene encodes nerve growth factor.

Another preferred embodiment of the present invention features a modified steroid hormone receptor protein. This protein is capable of binding a non-natural ligand. The modified receptor contains a steroid hormone receptor region which comprises a DNA binding domain, a mutated transregulatory domain and a mutated ligand binding domain. The mutated transregulatory domains are capable of transactivating gene expression but not transrepressing gene expression. Preferably the protein activates target gene expression and the target gene encodes nerve growth factor or functional equivalents thereof.

Examples of the mutated transregulatory domains are described in PCT Publication PCT/US96/04324, the whole of which (including drawings) is hereby incorporated by reference.

These modified receptors can be expressed by specially designing DNA expression vectors to control the level of expression of recombinant gene products. The steroid receptor family of gene regulatory proteins is an ideal set of such molecules. These proteins are ligand activated transcription factors whose ligands can range from steroid to retinoids, fatty acids, vitamins, thyroid hormones and other presently unidentified small molecules. These compounds bind to receptors and either activate or repress transcription.

These receptors are modified to allow them to bind various ligands whose structure is either naturally occurring or differs from naturally occurring ligands. By screening receptor mutants, receptors can be selected that respond to ligands which do not activate the host cell endogenous receptor. Thus, regulation of a desired transgene can be achieved using a ligand which binds to and regulates a customized receptor. This occurs only with cells that have incorporated and express the modified receptor.

Taking advantage of the abilities of the modified steroid hormone receptor to effect regulation of gene expression, these gene constructs can be used as therapeutic gene medicines, for gene replacement, and in gene therapy. These modified receptors are useful in gene therapy where the level of expression of a gene, whether transactivation or repression, is required to be controlled. The number of diseases associated with inappropriate production or responses to hormonal stimuli highlights the medical and biological importance of these constructs.

In addition, the constructs above can be used for gene replacement therapy in humans and for creating transgenic animal models used for studying human diseases. The transgenic models can be used as well for assessing and exploring novel therapeutic avenues to treat effects of chemical and physical carcinogens and tumor promoters. The above constructs can also be used for distinguishing steroid hormone receptor antagonists and steroid hormone receptor agonists. Such recognition of antagonist or agonist activity can be performed using cells transformed with the above constructs.

A preferred embodiment of the present invention features methods for transforming a cell with a vector containing nucleic acid encoding for a modified steroid hormone receptor. This method includes the steps of transforming a cell in situ by contacting the cell with the vector for a sufficient amount of time to transform the cell. As discussed above, transformation can be in vivo or ex vivo. Once transformed the cell expresses the mutated steroid hormone receptor. This method includes methods of introducing and methods of incorporating the vector. “Incorporating” and “introducing” as used herein refer to uptake or transfer of the vector into a cell such that the vector can express the therapeutic gene product within a cell as discussed with transformation above.

Other and further objects, features and advantages will be apparent from the following description of the presently preferred embodiments of the invention which are given for the purposes of disclosure when taken in conjunction with the accompanying drawings.

### BRIEF DESCRIPTION OF THE DRAWINGS

FIG. 1 shows the mutagenesis and screening strategy used in the present experiments.

FIG. 2 illustrates the functional and structural characterization of the UP-1 mutant.

FIG. 3 shows a western analysis of the mutant human progesterone receptor.

FIG. 4 shows the transcriptional activity and hormone binding analysis of wild type and mutant human progesterone receptor constructs.

FIG. 5 shows the specificity of transcriptional activity of the mutant human progesterone receptor.

FIG. 6 depicts the results of transient transfection of mutant human progesterone human receptor into mammalian cells.

FIG. 7 is a schematic representation of the gene switch.

FIG. 8 is a schematic representation of GLVP and its derivatives containing an additional transactivation domain.

FIG. 9 is a schematic representation of the effect of various lengths of poly-Q insertion on GLVP transactivation potential.

FIG. 10 is a schematic representation that an additional copy of the VP16 activation domain into GLVP does not further increase its transactivation potential.

FIG. 11 is a diagram of the original chimeric GLVP and its C-terminally extended derivatives.

FIG. 12 is a diagram of the transcriptional activation of GLVP versus its C-terminally located VP16 activation domain and various extensions of the hPR-LBD.

FIG. 13 is a diagram of the inducible repressors and reporters constructs.

The drawings are not necessarily to scale. Certain features of the invention may be exaggerated in scale or shown in schematic form in the interest of clarity and conciseness.

### DETAILED DESCRIPTION OF THE INVENTION

It will be readily apparent to one skilled in the art that various substitutions and modifications may be made to the invention disclosed herein without departing from the scope and spirit of the invention.

Definitions:

The term “steroid hormone receptor superfamily” as used herein refers to the superfamily of steroid receptors, some of which are known steroid receptors whose primary sequence suggests that they are related to each other. Representative examples of such receptors include the estrogen, progesterone, glucocorticoid-α, glucocorticoid-β, mineralocorticoid, androgen, thyroid hormone, retinoic acid, retinoid X, Vitamin D, COUP-TF, ecdysone, Nurr-1 and orphan receptors.

The term “progesterone receptor” as used herein also refers to a steroid hormone receptor which responds to or is activated by the hormone progesterone. Progesterone is part of the steroid hormone receptor superfamily as described above. The progesterone receptor can exist as two distinct but related forms that are derived from the same gene. The process for generation of the products may be alternate initiation of transcription, splicing differences, or transcription termination. These receptors are composed of DNA binding, ligand binding, as well as transregulatory domains. The progesterone receptor is also a ligand-dependent transcription factor capable of regulating gene expression. Interaction of the progesterone receptor with a ligand induces a cascade of molecular events that ultimately lead to the specific association of the activated receptor with regulatory elements of target genes.

Receptors are composed of a DNA binding domain and a ligand binding domain. The DNA binding domain contains the receptor regulating sequence and binds DNA and the ligand binding domain binds the specific biological compound (ligand) to activate the receptor.

The term “orphan receptors” as used herein refers to a family of approximately twenty receptors whose primary amino acid sequence is closely related to the primary amino acid sequence of the steroid hormone receptor. They are called orphan receptors because no ligand has been identified which directly activates any of the members of this family.

“A and B forms of the progesterone receptor” are two distinct forms of the progesterone receptor that are derived from the same gene. The process for generation of the products may be alternate initiation of transcription, splicing differences or may relate to the promotor structure.

The term “fusion protein” as used herein refers to a protein which is composed of two or more proteins (or fragments thereof) where each protein occurs separately in nature. The combination can be between complete amino acid sequences of the protein as found in nature, or fragments thereof.

“Estrogen response element” is a synthetic or naturally occurring DNA sequence which, when placed into a heterologous promotor can confer estrogen responsiveness to that promotor in the presence of estrogen activated estrogen receptor.

The term “ligand” refers to any compound which activates the receptor, usually by interaction with (binding) the ligand binding domain of the receptor. However, ligand can also include compounds which activate the receptor without binding.

“Agonist” is a compound which interacts with the steroid hormone receptor to promote a transcriptional response. Example estrogen is an agonist for the estrogen receptor, compounds which mimic estrogen would be defined as steroid hormone receptor agonists.

“Antagonist” is a compound which interacts with or binds to a steroid hormone receptor and blocks the activity of a receptor agonist.

The term “non-natural ligands” refer to compounds which are normally not found in animals or humans and which bind to the ligand binding domain of a receptor.

The term “anti-hormones” refers to compounds which are receptor antagonists. The anti-hormone is opposite in activity to a hormone.

The term “binding” or “bound” as used herein refers to the association, attaching, connecting, or linking through covalent or non-covalent means of a ligand, whether non-natural or natural with a corresponding receptor. The ligand and receptor interact at complementary and specific within sites on a given structure. Binding includes, but is not limited to, components which associate by electrostatic binding, hydrophobic binding, hydrogen binding, intercalation or forming helical structures with specific sites on nucleic acid molecules.

The term “non-native ligands” refers to those ligands which are not naturally found in the specific organism (man or animal) in which gene therapy is contemplated. For example, certain insect hormones such as ecdysone are not found in humans. This is an example of a non-native hormone to the human or animal.

The term “non-natural ligand” as used herein refers to compounds which can normally bind to the ligand binding domain of a receptor, but are not the endogenous ligand. “Endogenous” as used herein refers to a compound originating internally within mammalian cells. The receptor is not exposed to the ligand unless it is exogenously supplied. “Exogenous” as used herein refers to a compound originating from external sources and not normally present within mammalian cells. This also includes ligands or compounds which are not normally found in animals or humans. Non-natural also includes ligands which are not naturally found in the specific organism (man or animal) in which gene therapy is contemplated. These ligands activate receptors by binding to the modified ligand binding domain. Activation can occur through a specific ligand-receptor interaction whether it is through direct binding or through association in some form with the receptor.

“Natural ligand” as used here refers to compounds which normally bind to the ligand binding domain of a receptor and are endogenous. The receptor in this case is exposed to the ligand endogenously. Natural ligands include steroids, retinoids, fatty acids, vitamins, thyroid hormones, as well as synthetic variations of the above. This is meant to be only an example and nonlimiting.

The term “ligand” as referred to herein means any compound which activates the receptor, usually by interaction with the ligand binding domain of the receptor. Ligand includes a molecule or. an assemblage of molecules capable of specifically binding to a modified receptor. The term “specifically binding” means that a labeled ligand bound to the receptor can be completely displaced from the receptor by the addition of unlabeled ligand, as is known in the art.

Examples of non-natural ligands and non-native ligands may be found in PCT Publication PCT/US96/04324, the whole of which (including drawings) is hereby incorporated by reference.

Examples of non-natural ligands, anti-hormones and non-native ligands include the following: 11β-(4-dimethylaminophenyl)-17β-hydroxy-17α-propinyl-4,9-estradiene-3-one (RU38486 or Mifepristone or also known as RU486); 11β-(4-dimethylaminophenyl)-17α-hydroxy-17β-(3-hydroxypropyl)-13α-methyl-4,9-gonadiene-3-one (ZK98299 or Onapristone); 11β(4-acetylphenyl)-17β-hydroxy-17α-(1-propinyl)-4,9-estradiene-3-one (ZK112993); 11β-(4-dimethylaminophenyl)-17β-hydroxy-17α-(3-hydroxy-1(Z)-propenyl-estra-4,9-diene-3-one (ZK98734); (7β,11β,17β)-11-(4-dimethylaminophenyl)-7-methyl-4′,5′-dihydrospiro[ester-4,9-diene-17,2′(3′H)-furan]-3-one (Org31806); (11β,14β,17α)-4′,5′-dihydro-11-(4-dimethylaminophenyl)-[spiroestra-4,9-diene-17,2′(3′H)-furan]-3-one, (Org31376); 5-alpha pregnane-3, 20-dione. Examples of non-natural ligands and non-native ligands may be found in PCT Publication-PCT/US96/04324, the whole of which (including drawings) is hereby incorporated by reference.

The term “ligand binding domain” or “ligand binding region” as used herein refers to that portion of a steroid hormone receptor protein which binds the appropriate hormone or ligand and induces a cascade of molecular events that ultimately leads to the specific association of the activated receptor with regulatory elements of target genes. This includes, but is not limited to, the positive or negative effects on regulation of gene transcription. Binding of ligand to the ligand binding domain induces a conformation change in the receptor structure. The conformational change includes the dissociation of heat shock proteins and the release of a monomeric receptor from the receptor complex, as well as a different tertiary or 3-dimensional structure. The conformational change that occurs is specific for the steroid receptor and ligand that binds to the ligand binding domain.

The term “DNA binding domain” as used herein refers to that part of the steroid hormone receptor protein which binds specific DNA sequence in the regulatory regions of target genes. This domain is capable of binding short nucleotide stretches arranged as palindromic, inverted or repeated repeats. Such binding, will activate gene expression depending on the specific ligand and the confonnational changes due to such ligand binding. For repression, DNA binding is not needed.

The term “transregulatory domain” as used herein refers to those portions of the steroid hormone receptor protein which are capable of transactivating or transrepressing gene expression. This would include different regions of the receptor responsible for either repression or activation, or the regions of the receptor responsible for both repression and activation. Such regions are spatially distinct. The above is only an example and meant to be non-limiting. For transrepression, this domain under one mechanism is involved with dimerization which in turn causes a protein/protein interaction to prevent or repress gene expression. Such regulation occurs when the receptor is activated by the ligand binding to the ligand binding domain. The conformational change of the receptor is capable of forming a dimer with a discrete portion of the transregulatory domain to repress gene expression. In addition, repression can occur through a monomeric form of the receptor, however, DNA binding is not necessary (see below).

The terms “transactivation,” “transactivate,” or “transactivating” refer to a positive effect on the regulation of gene transcription due the interaction of a hormone or ligand with a receptor causing the cascade of molecular events that ultimately lead to the specific association of the activated receptor with the regulatory elements of the target genes. Transactivation can occur from the interaction of non-natural as well as natural ligands. Agonist compounds which interact with steroid hormone receptors to promote transcriptional response can cause transactivation. Such positive effects on transcription include the binding of an activated receptor to specific recognition sequences in the promoter of target genes to activate transcription. The activated receptors are capable of interacting specifically with DNA. The hormone- or ligand-activated receptors associate with specific DNA sequences, or hormone response elements, in the regulatory regions of target genes. Transactivation alters the rate of transcription or induces the transcription of a particular gene(s). It refers to an increase in the rate and/or amount of transcription taking place.

The terms “transrepress,” “transrepression” or “transrepressing” as used herein refer to the negative effects on regulation of gene transcription due to the interaction of a hormone or ligand with a receptor inducing a cascade of molecular events that ultimately lead to the specific association of the activated receptor with other transcription factors such as NF_{K}-B or AP-1. Transrepression can occur from the interaction of non-natural as well as natural ligands. Antagonist and agonist compounds which interact with steroid hormone receptor can cause transrepression. Once the ligand binds to the receptor, a conformational change occurs. Transrepression can occur via two different mechanisms, i.e., through a dimeric and monomeric form of the receptor. Use of the monomeric form of the receptor for transrepression depends on the presence of the DNA binding domain but not on the ability of the receptor to bind DNA. Use of the dimeric form of the receptor for transrepression depends on the receptor binding response elements overlapping cis-elements(s). Transrepression alters the rate of transcription or inhibits the transcription of a particular gene. Transrepression decreases the rate and/or the amount of transcription taking place.

The term “genetic material” as used herein refers to contiguous fragments of DNA or RNA. The genetic material which is introduced into targeted cells according to the methods described herein can be any DNA or RNA. For example, the nucleic acid can be: (1) normally found in the targeted cells, (2) normally found in targeted cells but not expressed at physiologically appropriate levels in targeted cells, (3) normally found in targeted cells but not expressed at optimal levels in certain pathological conditions, (4) not normally found in targeted cells, (5) novel fragments of genes normally expressed or not expressed in targeted cells, (6) synthetic modifications of genes expressed or not expressed within targeted cells, (7) any other DNA which may be modified for expression in targeted cells and (8) any combination of the above.

The term “gene expression” or “nucleic acid expression” as used herein refers to the gene product of the genetic material from the transcription and translation process. Expression includes the polypeptide chain translated from an mRNA molecule which is transcribed from a gene. If the RNA transcript is not translated, e.g., rRNA, tRNA, the RNA molecule represents the gene product.

The term “nucleic acid sequence,” “gene,” “nucleic acid” or “nucleic acid cassette” as used herein refers to the genetic material of interest which can express a protein, or a peptide, or RNA after it is incorporated transiently, permanently or episomally into a cell. The nucleic acid cassette is positionally and sequentially oriented in a vector with other necessary elements such that the nucleic acid in the cassette can be transcribed and, when necessary, translated in the cells.

The term “modified,” “modification,” “mutant” or “mutated” refers to an alteration of the primary sequence of a receptor such that it differs from the wild type or naturally occurring sequence. The mutant steroid hormone receptor protein as used in the present invention can be a mutant of any member of the steroid hormone receptor superfamily. For example, a steroid receptor can be mutated by deletion of amino acids on the carboxy terminal end of the protein. Generally, a deletion of from about 1 to about 120 amino acids from the carboxy terminal end of the protein provides a mutant useful in the present invention. A person having ordinary skill in this art will recognize, however, that a shorter deletion of carboxy terminal amino acids will be necessary to create useful mutants of certain steroid hormone receptor proteins. For example, a mutant of the progesterone receptor protein will contain a carboxy terminal amino acid deletion of from about 1 to about 60 amino acids. In a preferred embodiment 42 carboxy terminal amino acids are deleted from the progesterone receptor protein. Examples of mutations are described in PCT Publication PCTIUS96/04324, the whole of which (including drawings) is hereby incorporated by reference.

One skilled in the art will recognize that a combination of mutations and/or deletions are possible to gain the desired response. This would include double point mutations to the same or different domains. In addition, mutation also includes “null mutations” which are genetic lesions to a gene locus that totally inactivate the gene product. “Null mutation” is a genetic lesion to a gene locus that totally inactivates the gene product.

The term mutation also includes any other derivatives. The term “derivative” as used herein refers to a peptide or compounds produced or modified from another peptide or compound of a similar structure. Such a derivative may be a “chemical derivative,” “fragment,” “variant,” chimera,” or “hybrid” of the complex. A derivative retains at least a portion of the function of the protein (for example reactivity with an antibody specific for the complex, enzymatic activity or binding activity medicated through noncatalytic domains) which permits its utility in accordance with the present invention.

A derivative may be a complex comprising at least one “variant” polypeptide which either lacks one or more amino acids or contains additional or substituted amino acids relative to the native polypeptide. The variant may be derived from a naturally occurring complex component by appropriately modifying the protein DNA coding sequence to add, remove, and/or to modify codons for one or more amino acids at one or more sites of the C-terminus, N-terminus, and/or within the native sequence. It is understood that such variants having added, substituted and/or additional amino acids retain one or more characterizing portions of the native complex. A functional derivative of complexes comprising proteins with deleted, inserted and/or substituted amino acid residues may be prepared using standard techniques well-known to those of ordinary skill in the art.

A “chemical derivative” of the complex contains additional chemical moieties not normally a part of the protein. Such moieties may improve the molecule's solubility, absorption, biological half life, and the like.

The term “plasmid” as used herein refers to a construction comprised of extrachromosomal genetic material, usually of a circular duplex of DNA which can replicate independently of chromosomal DNA. Plasmids are used in gene transfer as vectors. Plasmids which are helpful in the present invention include plasmids selected from the group consisting of UP-1, YEphPR-A879, YEphPR-A891, YEphPR-B891, YEphPR-B879, phPR-A879, phPR-A891, phPR-B879 and phPR-B891.

The term “vector” as used herein refers to a construction comprised of genetic material designed to direct transformation of a targeted cell. A vector contains multiple genetic elements positionally and sequentially oriented with other necessary elements such that the nucleic acid in a nucleic acid cassette can be transcribed and when necessary translated in the transfected cells. The term vector as used herein can refer to nucleic acid, e.g., DNA derived from a plasmid, cosmid, phagemid or bacteriophage, into which one or more fragments of nucleic acid may be inserted or cloned which encode for particular proteins. The term “plasmid” as used herein refers to a construction comprised of extrachromosomal genetic material, usually of a circular duplex of DNA which can replicate independently of chromosomal DNA. The plasmid does not necessarily replicate.

The vector can contain one or more unique restriction sites, and may be capable of autonomous replication in a defined host or organism such that the cloned sequence is reproduced. The vector molecule can confer some well-defined phenotype on the host organism which is either selectable or readily detected. The vector may have a linear or circular configuration. The components of a vector can contain but is not limited to a DNA molecule incorporating: (1) DNA; (2) a sequence encoding a therapeutic or desired product; and (3) regulatory elements for transcription, translation, RNA processing, RNA stability, and replication.

The purpose of the vector is to provide expression of a nucleic acid sequence in cells or tissue. Expression includes the efficient transcription of an inserted gene or nucleic acid sequence. Expression products may be proteins, polypeptides, or RNA. The nucleic acid sequence can be contained in a nucleic acid cassette. Expression of the nucleic acid can be continuous, constitutive, or regulated. The vector can also be used as a prokaryotic element for replication of plasmid in bacteria and selection for maintenance of plasmid in bacteria.

In one embodiment the vector comprises the following elements linked sequentially at an appropriate distance to allow functional expression: a promoter, a 5′ mRNA leader sequence, a translation initiation site, a nucleic acid cassette containing the sequence to be expressed, a 3′ mRNA untranslated region, and a polyadenylation signal sequence. As used herein the term “expression vector” refers to a DNA vector that contains all of the information necessary to produce a recombinant protein in a heterologous cell.

In addition, the term “vector” as used herein can also include viral vectors. A “viral vector” in this sense is one that is physically incorporated in a viral particle by the inclusion of a portion of a viral genome within the vector, e.g., a packaging signal, and is not merely DNA or a located gene taken from a portion of a viral nucleic acid. Thus, while a portion of a viral genome can be present in a vector of the present invention, that portion does not cause incorporation of the vector into a viral particle and thus is unable to produce an infective viral particle.

A vector as used herein can also include DNA sequence elements which enable extra-chromosomal (episomal) replication of the DNA. Vectors capable of episomal replication are maintained as extra-chromosomal molecules and can replicate. These vectors are not eliminated by simple degradation but continue to be copied. These elements may be derived from a viral or mammalian genome. These provide prolonged or “persistent” expression as described below.

The term “vehicle” as used herein refers to non-genetic material combined with the vector in a solution or suspension which enhances the uptake, stability and expression of genetic material into targeted cells. Examples of a vehicle include: sucrose, protamine, polybrene, spermidine, polylysine, other polycations, proteins, CaPO₄ precipitates, soluble and insoluble particles, or matrices for slow release of genetic material. The proteins may be selected from the group including lactoferrin, histone, natural or synthetic DNA binding proteins, natural or synthetic DNA binding compounds, viral proteins, non-viral proteins or any combinations of these. In addition, vehicles may be comprised of synthetic compounds which bind both to DNA and function as ligands for normal receptors on targeted cells.

The term “transformed” as used herein refers to transient, stable or persistent changes in the characteristics (expressed phenotype) of a cell by the mechanism of gene transfer. Genetic material is introduced into a cell in a form where it expresses a specific gene product or alters the expression or effect of endogenous gene products. One skilled in the art readily recognizes that the nucleic acid cassette can be introduced into the cells by a variety of procedures, including transfection and transduction.

The term “transfection” as used herein refers to the process of introducing a DNA expression vector into a cell. Various methods of transfection are possible including microinjection, CaPO₄ precipitation, liposome fusion (e.g. lipofection), electroporation, or use of a gene gun. Those are only examples and are meant not to be limiting. The term “transfection” as used herein refers to the process of introducing DNA (e.g., DNA expression vector) into a cell.

Following entry into the cell, the transfected DNA may: (1) recombine with the genome of the host; (2) replicate independently as an episome; or (3) be maintained as an episome without replication prior to elimination. Cells may be naturally able to uptake DNA. Particular cells which are not naturally able to take up DNA require various treatments, as described above, in order to induce the transfer of DNA across the cell membrane.

The term “transduction” as used herein refers to the process of introducing recombinant virus into a cell by infecting the cell with a virus particle. In the present invention, the recombinant virus contains a nucleic acid cassette.

The term “transient” as used herein relates to the introduction of genetic material into a cell to express specific proteins, peptides, or RNA, etc. The introduced genetic material is not integrated into the host cell genome or replicated and is accordingly eliminated from the cell over a period of time.

The term “stable” as used herein refers to the introduction of genetic material into the chromosome of the targeted cell where it integrates and becomes a permanent component of the genetic material in that cell. Gene expression after stable transduction can permanently alter the characteristics of the cell leading to stable transformation.

An episomal transformation is a variant of stable transformation in which the introduced gene is not incorporated in the host cell chromosomes but rather is replicated as an extrachromosomal element. This can lead to apparently stable transformation of the characteristics of a cell. “Transiently” as used herein refers to the introduction of a gene into a cell to express the nucleic acid, e.g., the cell expresses specific proteins, peptides or RNA, etc. The introduced gene is not integrated into the host cell genome and is accordingly eliminated from the cell over a period of time. Transient expression relates to the expression of a gene product during a period of transient transfection. Transient expression also refers to transfected cells with a limited life span.

Transformation can be performed by in vivo techniques or ex vivo techniques as described in PCT Publication PCT/US96/04324, the whole of which (including drawings) is hereby incorporated by reference. Transformation can be tissue specific to regulate expression of the nucleic acid predominantly in the tissue or cell of choice.

Transformation of the cell may be associated with production of a variety of gene products including protein and RNA. Such products are described in PCT Publication PCTIUS96/04324, the whole of which (including drawings) is hereby incorporated by reference. The product expressed by the transformed cell depends on the nucleic acid of the nucleic acid cassette. In the present invention the nucleic acid to be expressed is a fusion protein as referenced above, or variations thereof or any of the other receptor proteins disclosed herein.

The term “persistent” as used herein refers to the introduction of genes into the cell together with genetic elements which enable episomal (extrachromosomal) replication. This can lead to apparently stable transformation of the characteristics of the cell without the integration of the novel genetic material into the chromosome of the host cell.

The present invention features methods for administration as discussed above. Such methods include methods for administering a supply of polypeptide, protein or RNA to a human, animal or to tissue culture or cells. These methods of use of the above-referenced vectors comprises the steps of administering an effective amount of the vectors to a human, animal or tissue culture. The term “administering” or “administration” as used herein refers to the route of introduction of a vector or carrier of DNA into the body. The vectors of the above methods and the methods discussed below may be administered by various routes. Administration may be intravenous, intratissue injection, topical, oral, or by gene gun or hypospray instrumentation. Administration can be directly to a target tissue, e.g. direct injection into synovial cavity or cells, or through systemic delivery. These are only examples and are nonlimiting.

Administration will include a variety of methods, as described in PCT Publication PCT/US96/04324, the whole of which (including drawings) is hereby incorporated by reference. See, also, WO 93/18759, the whole of which is hereby incorporated by reference. The preferred embodiment is by direct injection. Routes of administration include intramuscular, aerosol, oral, topical, systemic, ocular, intraperitoneal, intrathecal and/or fluid spaces.

In one embodiment the transformed cell is a muscle cell. The term “muscle” refers to myogenic cells including myoblasts, skeletal, heart and smooth muscle cells. The muscle cells or tissue can be in vivo, in vitro or tissue culture and capable of differentiating into muscle tissue. In another embodiment, the transformed cell is a lung cell. The term “lung cell” as used herein refers to cells associated with the pulmonary system. The lung cell can also be in vivo, in vitro or tissue culture.

In still another embodiment, the transformed cell is a cell associated with the joints. The term “cells associated with the joints” refers to all of the cellular and non-cellular materials which comprise the joint (e.g., knee or elbow) and are involved in the normal function of the joint or are present within the joint due to pathological conditions. These include material associated with: the joint capsule such as synovial membranes, synovial fluid, synovial cells (including type A cells and type B synovial cells); the cartilaginous components of the joint such as chondrocyte, extracellular matrix of cartilage; the bony structures such as bone, periosteum of bone, periosteal cells, osteoblast, osteoclast; the immunological components such as inflammatory cells, lymphocytes, mast cells, monocytes, eosinophil; other cells like fibroblasts; and combinations of the above. Once transformed these cells express the fusion protein. One skilled in the art will quickly realize that any cell is capable of undergoing transformation and within the scope of this invention.

The term “effective amount” as used herein refers to sufficient vector administered to humans, animals or into tissue culture cells to produce the adequate levels of polypeptide, protein, or RNA. One skilled in the art recognizes that the adequate level of protein polypeptide or RNA will depend on the intended use of the particular vector. These levels will be different depending on the type of administration, treatment or vaccination as well as intended use.

The term “pharmacological dose” as used herein with a vector/molecular switch complex refers to a dose of vector and level of gene expression resulting from the action of the promoter on the nucleic acid cassette when introduced into the appropriate cell type which will produce sufficient protein, polypeptide, or antisense RNA to either (1) increase the level of protein production, (2) decrease or stop the production of a protein, (3) inhibit the action of a protein, (4) inhibit proliferation or accumulation of specific cell types, or (5) induce proliferation or accumulation of specific cell types. The dose will depend on the protein being expressed, the promoter, uptake and action of the protein RNA. Given any set of parameters, one skilled in the art will be able to determine the dose.

The term “pharmacological dose” as used herein with a ligand refers to a dose of ligand sufficient to cause either up-regulation or down-regulation of the nucleic acid cassette. Thus, there will be a sufficient level of ligand such that it will bind with the receptor in the appropriate cells in order to regulate the nucleic acid cassette. The specific dose of any ligand will depend on the characteristics of the ligand entering the cell, binding to the receptor and then binding to the DNA and the amount of protein being expressed and the amount of up-regulation or down-regulation needed. Given any set of parameters, one skilled in the art will be able to determine the appropriate dose for any given receptor being used as a molecular switch.

“Plasmid activity” is a phenotypic consequence that relates specifically to introduction of a plasmid into an assay system.

“Transcriptional activity” is a relative measure of the degree of RNA polymerase activity at a particular promotor.

“Receptor activity” is a phenotypic consequence that relates specifically to introduction of a receptor into an assay system.

By “transgenic animal” is meant an animal whose genome contains an additional copy or copies of the gene from the same species or it contains the gene or genes of another species, such as a gene encoding for a mutated glucocorticoid receptor introduced by genetic manipulation or cloning techniques, as described herein and as known in the art. The transgenic animal can include the resulting animal in which the vector has been inserted into the embryo from which the animal developed or any progeny of that animal. The term “progeny” as used herein includes direct progeny of the transgenic animal as well as any progeny of succeeding progeny. Thus, one skilled in the art will readily recognize that if two different transgenic animals have been made each utilizing a different gene or genes and they are mated, the possibility exists that some of the resulting progeny will contain two or more introduced genes. One skilled in the art will readily recognize that by controlling the matings, transgenic animals containing multiple introduced genes can be made.

Preferred Embodiments

The present invention provides mutant steroid hormone receptor proteins. These mutated steroid hormone receptor proteins are capable of distinguishing, and are useful in methods of distinguishing a steroid hormone receptor antagonist from a steroid hormone receptor agonist.

The present invention further provides plasmids containing mutated steroid hormone receptor proteins. Plasmids of the present invention may contain mutant proteins of any of the hormones in the steroid hormone receptor superfamily.

The present invention also provides transfected cells containing plasmids having mutated steroid hormone receptor proteins inserted therein. Useful cells for transfection include yeast, mammalian and insect cells.

In a specific embodiment, the yeast is Saccharomyces cerevisiae. In a specific embodiment the mammalian cell is selected from the group consisting of HeLa, CV-1, COSM6, HepG2, CHO and Ros 17.2. In a specific embodiment the insect cells are usually selected from the group consisting of SF9, drosophilia, butterfly and bee.

The present invention also provides stable cell lines transformed with the plasmids of the present invention.

The plasmids and transfected cells of the present invention are useful in methods of determining whether a compound has antagonist or agonist activity at a steroid hormone receptor. This method comprises contacting a compound of interest with a transfected cell of the present invention. If the compound induces transcription, it has a steroid hormone receptor antagonist. If no transcription is induced, the compound may be a steroid hormone receptor agonist.

The present invention also provides a method of determining an endogenous ligand for a steroid hormone receptor protein. This method comprises initially contacting a compound with a transfected cell of the present invention. Subsequently, the transcription level induced by the compound is measured. The higher the transcription level the more strongly the indication that the compound is an endogenous ligand of the specific receptor being tested.

In addition, the present invention provides endogenous ligands for steroid hormone receptor proteins. An endogenous ligand for a steroid hormone receptor protein is capable of stimulating transcription when in the presence of a transfected cell of the present invention. The endogenous ligand binds to the mutated steroid receptor of the present invention and stimulates transcription in cells containing the mutated receptor.

Another alternative embodiment of the present invention is a molecular switch for regulating expression of a heterologous nucleic acid sequence in gene therapy.

In a preferred embodiment of the present invention, the molecular switch for regulating expression of a heterologous nucleic acid cassette in gene therapy, comprises a modified steroid receptor which includes a natural steroid receptor DNA binding domain attached to a modified ligand binding domain. In the preferred embodiment of the molecular switch the modified binding domain usually binds only ligand compounds which are non-natural ligands, anfi-hormones or non-native ligands. One skilled in the art readily recognizes that the modified ligand binding domain may bind native ligands, but there is insignificant binding and thus very little, if any, regulation.

In a preferred embodiment, the modified steroid receptor is a progesterone receptor with the DNA binding domain replaced with a DNA binding domain selected from the group consisting of GAL-4 DNA, virus DNA binding site, insect DNA binding site and a non-mammalian DNA binding site.

The molecular switch can be further modified by the addition of a transactivation domain. The transactivation domains which are usually used include VP-16, TAF-1, TAF-2, TAU-1 and TAU-2. One skilled in the art will readily recognize that a variety of other transactivation domains are available.

In a preferred embodiment the progesterone receptor has the modified ligand binding domain GAL-4 DNA binding domain and a transactivation domain such as TAF-1.

In a further embodiment, the progesterone receptor has the ligand binding domain replaced with an ecdysone binding domain. Again, the function of this molecular switch can be enhanced by adding a TAF-1 transactivation domain.

One skilled in the art will readily recognize the molecular switch can be made tissue specific by selecting the appropriate transactivation domains, ligand binding domains and DNA binding domains. In particular, one skilled in the art readily recognizes that by adding a transactivation domain which is specific to a given tissue, the molecular switch will only work in that tissue. Also, the addition of a tissue-specific cis-element to the target gene will aid in providing tissue-specific expression.

The present invention also envisions a method of regulating gene expression of a nucleic acid cassette in gene therapy. This method comprises the step of attaching the molecular switch to a nucleic acid cassette used in gene therapy. In the preferred embodiment, the nucleic acid sequence which is expressed is heterologous. The combined nucleic acid cassette/molecular switch is then administered in a pharmacological dose to an animal or human to be treated or to a transgenic animal or to a plant.

One skilled in the art readily appreciates that the combined nucleic acid cassette/molecular switch can be introduced into the cell in a variety of ways both in vivo and ex vivo. The introduction can be by transfection or transduction. After the nucleic acid cassette/molecular switch is introduced into the cell, the cassette in the resultant transformed cell can be either up-regulated (turned on) or down-regulated (turned off) by introducing to the animal or human a pharmacological dose of a ligand which binds the modified ligand binding site.

In one embodiment of the present invention there is a method for regulating nucleic acid cassette expression in gene therapy comprising the step of linking a molecular switch to a nucleic acid cassette. This molecular switch/nucleic acid cassette is introduced into a cell to form a transformed cell. The transformed cell is then inserted in a pharmacological dose into a human or animal for gene therapy.

In another embodiment the molecular switch/nucleic acid cassette is directly injected into a targeted cell in vivo for gene therapy.

For example, in the treatment of senile dementia or Parkinson's disease, the nucleic acid within the nucleic acid cassette contains a growth factor, hormone or neurotransmitter and the cell is a brain cell. In a preferred embodiment the naked brain cell containing the cassette can be encapsulated in a permeable structure. The naked brain cell or the permeable structure containing the brain cell is then inserted into the animal or human to be treated. The permeable structure is capable of allowing the in/out passage of activators of the molecular switch and growth factors but prevents the passage of attack cells that would interact with and damage the implanted brain cells. In the preferred embodiment it is important to encapsulate the brain cells, since introduction of naked brain cells often results in attack by the body's defense system and the destruction of these cells. One skilled in the art recognizes that a variety of encapsulation procedures and structures are available in the art.

In the treatment of senile dementia or Parkinson's disease, it is found that the molecular switch in the preferred embodiment includes a progesterone receptor with the modified ligand binding domain replaced attached to a GAL-4 DNA. A growth factor is produced in the transformed cell by giving a pharmacological dose of an appropriate ligand to turn the molecular switch on (up-regulation) to the animal or human to be treated. For example, an anti-progesterone such as RU38486 (or also known as RU486) can be given. The amount of growth factor produced is proportional to the dose of ligand given. One skilled in the art will be able to determine a pharmacological dose depending on the molecular switch used and the ligand used.

Another embodiment of the present invention employs a dual system of agonist/antagonist pairs. In this system a custom up-regulation ligand is chosen and the desired receptor mutation or modified receptors are made. Then a second round of ligand screening and mutation is performed to develop a receptor which also binds a specific, selective down-regulator ligand. In the preferred embodiment the ligands share a normal metabolic clearance pathway of the host's endogenous ligands, thereby avoiding problems of toxicity and long half-life. In the screening process either yeast, animal or insect cells can be used. In the preferred embodiment yeast cells are used.

In addition to selecting transactivation elements and receptors for tissue specificity, one skilled in the art also recognizes that tissue specificity can be achieved with specific ligands. For example, ligands can be chosen which act only in certain tissues due to requirements for terminal conversion to active metabolites. A synthetic androgen which binds a transfected androgen receptor is made. This androgen, however, requires metabolism to the 5-alpha reduced form to be active. In this manner only classical androgen end-organs are able to metabolize the new ligand to its proper chemical form. Other cells of the body lacking the 5-alpha reductase will not activate the transgene via this compound.

Alternatively, a ligand which is active only when it is not further metabolized to the 5-alpha reduced form is used. In this case, the ligand would be active only in classical androgen end-organ cells. Since 5-alpha reductase inhibitors are currently available therapeutic agents, they can be used in conjunction with the present invention to allow complete shutdown or complete activation of the receptor bypassing the ligand route if some sort of emergency required that approach.

Side chains are usually tolerated at certain positions on ligands of the receptor superfamily. For example, the 7-alpha position of certain ligands, such as estradiol and progesterone, can be attached to sidechains and the ligands will still bind to receptors. Suitable sidechains can be used to either increase or restrict solubility, membrane transfer or target organ accessibility. Thus, even specific ligands can be made to show tissue preference. For example, the synthetic steroid R5020 (17α,21-dimethyl-19-Norpregna-4,9-diene-3,20-dione) does not enter tissue culture cells at low temperatures at which progesterone enters freely. One skilled in the art readily recognizes that other modifications can be made to ligands to tailor their use as up- or down-regulating agents in the present invention.

The present invention provides modified proteins of steroid hormone receptors. Steroid hormone receptors which may be modified include any of those receptors which comprise the steroid hormone receptor superfamily. Representative examples of such receptors include the estrogen, progesterone, glucocorticoid, mineralocorticoid, androgen, thyroid hormone, retinoic acid, retinoid X and Vitamin D3 receptors.

In another embodiment, the modified steroid hormone receptor proteins of the present invention include a steroid receptor region made up of a DNA binding domain, one or more transregulatory domains and a mutated steroid receptor ligand binding region capable of binding a non-natural ligand.

The DNA binding domain contains the receptor regulating sequence and binds DNA. Such a domain may be a yeast GAL4 DNA binding domain. The ligand binding domain binds the specific compound which will activate the receptor, for example RU486.

Several different functional domains have been characterized in transcription factors; they can be either acidic (VP16, GAL4), glutamine-rich (SP1, Oct-1, Oct2A), proline-rich (Oct3/4), or serine- and threonine-rich (Pit1) (Wegner et al., Curr. Opin. in Cell Biol. 5:488-498 (1993)). It is known that different types of transcriptional activation domains interact with different coactivators of the general transcriptional machinery. When different activation domains are fused together in a transactivator, they can synergize with each other to increase its transcriptional potential. Recently, Gerber, et al. demonstrated that insertion of either a poly-glutamine (poly-Q) or poly-proline (poly-P) stretch within the GAL4-VP16 enhances the activation of GAL4-VP16 (Gerber et al., Science 263:808-811 (1994)).

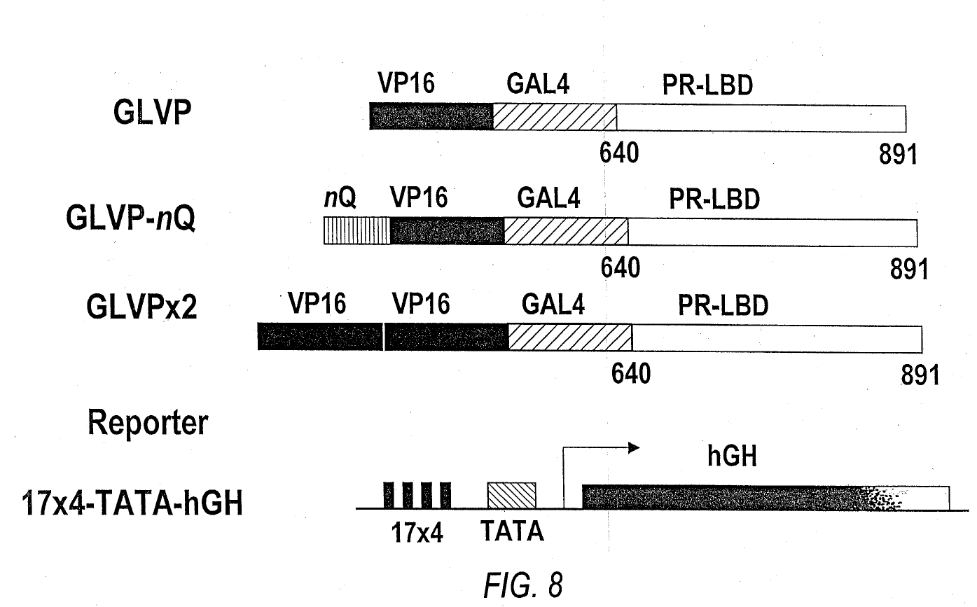

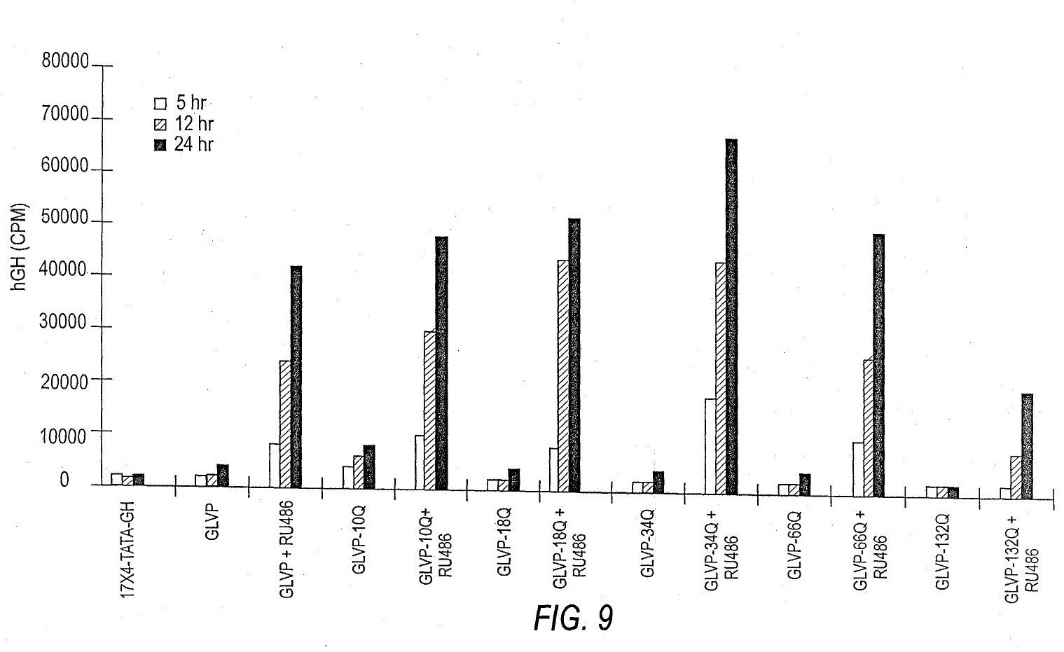

In order to increase the potency of the Gal-4-VP16-Progesterone Ligand Binding Domain (“GLVP”) regulator, varying lengths of poly-Q stretches encoded by the triplet repeats (CAG)n were inserted into the N-terminus of the GLVP regulator (FIG. 8). Transactivation analysis of the various sizes of poly-Q insertions in the GLVP indicate that addition of 10-34Q increases transcriptional activity of the regulator on the reporter gene (17x4-TATA-hGH), while further extension of poly-Q from a 66Q-oligomer to a 132Q-oligomer results in decreased activation of target gene (FIG. 9). These experiments demonstrated that a combination of different types of functional domains of appropriate strength further improves the activation potential of the GLVP chimeric regulator.

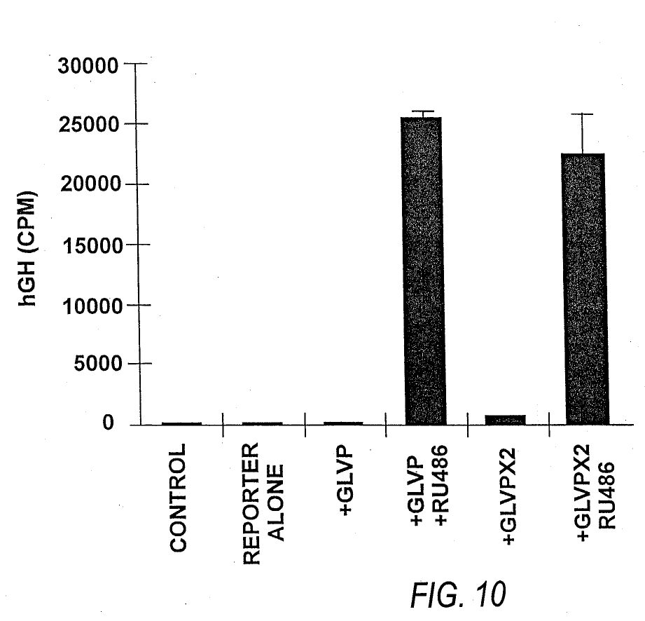

To understand whether additional activation domains of the same type would also increase the activation potential of the chimeric regulator, GLVPx2 with 2 copies of VP16 activation domain at the N-terminus was constructed (FIG. 8). As shown in FIG. 10, further addition of the same type of transactivation domain (VP16) did not increase the activation potential of the regulator.

The original GLVP can efficiently activate target gene expression containing stronger promoters such as the thymidine kinase (tk) promoter. To further enhance the transcriptional activity of GLVP, a more potent RU486-inducible gene regulator was generated. This new gene regulator responds to RU486 at a concentration even lower than that used by the original GLVP. At this concentration, RU486 does not have any anti-progesterone or anti-glucocorticoid activity. The inducible system has been used successfully to produce secreted NGF from a reporter gene in an RU486 dependent manner to induce neurite outgrowth in co-cultured PC12 cells (of rat adrenal pheochromocytoma). This RU486-controllable ligand binding domain can also be converted to an inducible repressor for shutting down target gene expression. Individual domains within a chimeric fusion protein have been shown to influence each other's function in a position-dependent context.

Transcriptional regulation of gene expression has been intensively studied over the past decade (McKnight, Genes Dev. 10:367-381; Goodrich et al., Curr. Opin. in Cell Biol. 6:403-409; Pugh, Curr. Opin. in Cell Biol. 8:303-311). It is generally believed that transcription factors selectively bind to their recognition sequences on DNA (promoters and enhancers) and directly interact with the TBP-associated factors (TAFs), coactivators, or corepressors to activate or repress transcriptional activity. Nuclear hormone receptors, such as steroid, thyroid, retinoid and orphan receptors, are an unique class of inducible transcription factors that can modulate their respective target genes in response to their cognate ligands. Recently, several coactivators (SRC-1) (Onate et al., Science 270:1354-1357 (1995)), CBP (Kamei et al., Cell 85:403-414 (1996)), and corepressors (N-CoR, SMART) (Hörlein et al., Nature 377:397-404 (1995); Chen et al., Nature 377:454-457 (1995)), that mediate nuclear hormone receptor activation of target genes have been identified. These studies suggest that multiple protein factors are involved in the complex process of transcriptional regulation of gene expression.

Mutagenesis studies of the hPR ligand binding domain have demonstrated that extension of the LBD deletion from amino acid position 891 to 914 increases the activation potential of the chimeric regulator. Addition of this short stretch of 23 amino acids increases the PR-LBD's dimerization potential and subsequent binding to its response element. Further extension of the hPR-LBD from residue 917 to 928 results in a decrease of transactivation, suggesting that this region may serve as a repressor interacting domain. In fact, when this 12 amino acid stretch is ligated to the GAL4 DNA binding domain, it is sufficient to confer transcriptional repression of a target gene, suggesting that these 12 amino acids might interact with a yet unidentified cellular co-repressor (Xu et al., (unpublished) (1996)).

Many chimeric proteins have been constructed in recent years in order to combine different functional domains of various proteins into one versatile chimera. While it is clear that each protein domain can function independently, relatively little is known about how individual domains modulate each other's function within a chimeric protein. The activation potential of VP16 is influenced by its relative position within the chimeric regulator. The C-terminally located VP16 chimeric regulator GL₉₁₄VP_{C′} effectively activates target gene expression containing a minimal promoter at an RU486 concentration 10-fold lower than its N-terminally located VP16 counterpart, GL₉₁₄VP. At this concentration, RU486 is expected to have no interference with endogenous gene expression.

This new inducible system will afford an improved margin of safety and further contribute to its application for gene regulation in vivo. Mutational studies revealed that the chimeric regulator GL₉₁₄VP_{C′} is about 8 to 10 times more potent than our originally described regulator GLVP and responds at a lower ligand concentration. Furthermore, within a chimeric protein, individual functional domains, such as those involved in transactivation, DNA binding and ligand binding, can modulate each other's function, depending on their relative positions.

Protein-protein interaction studies suggest that different types of transactivation or transrepression domains interact with their respective TAFs or coactivator, corepressor molecules within the RNA polymerase II preinitiation complex to alter gene transcription (Pugh, Curr. Opin. in Cell Biol. 8:303-311; Goodrich et al., Cell 75:519-530 (1993)). Glutamine rich stretches have been identified in various transcriptional factors (SP1, Oct-1 and androgen receptor) although their precise function is unknown (Wegner et al., Curr. Opin. in Cell Biol. 5:488-498 (1993); Gerber et al., Science 263:808-811 (1994)). Expanded regions of triplet CAG repeats have been implicated in several neurodegenerative diseases such as Huntington's, Kennedy's, dentatorubral-pallidoluysian atrophy (DRPLA), and hereditary spinocerebellar ataxias (SCA1) (Kuhl et al., Curr. Opin. in Genet. Dev. 3:404-407 (1993); Ross et al., Trends in Neurosci 16:254-260 (1993)); Ashley et al., Annu. Rev. Genet. 29:703-728 (1995)).

Recently, several groups have isolated proteins responsible for the above mentioned neurodegenerative diseases and confirmed that they indeed contain long poly-glutamine (Q) stretches encoded by the expanded CAG repeats (Servadio et al., Nature Genet. 10:94-98 (1995); Yazawa et al., Nature Genet. 10:99-103 (1995); Trottier et al., Nature Genet. 10:104-110 (1995)). To further understand the role of poly-Q stretches in transcriptional regulation, various lengths of poly-Q was inserted in the N-terminus of GLVP.

Addition of a 10-34 oligomer of poly-Q results in synergistic transcriptional activation, while expanded CAG triplet repeats beyond 66 oligomeric glutamines do not further increase the transactivation potential of chimeric regulator GLVP. These observations suggest that structural and conformational changes might be involved in proteins encoded by the expanded CAG triplet repeat as compared with the regular length poly-Q which encoded by 10-30 repeats of CAG in normal protein. These results suggest that a neurological disease with expanded CAG repeats (>40 mer) may not be due to aberrant high transcriptional potential but rather due to an influence on other aspects of cell function (Burke et al., Nature Medicine 2:347-350 (1996)).

A transcription factor can either activate or repress gene expression depending on the promoter/enhancer context of its particular target DNA and the coregulator proteins with which it interacts (Kingston et al. Genes Dev. 10:905-920 (1996)). For example, in the absence of thyroid hormone (T3), the thyroid hormone receptor (TR) normally binds to its recognition sequence on DNA and represses target gene activation through interactions with corepressors (Baniahmad et al., Mol. Cell. Biol. 15:76-86 (1995); Shibata et al. (unpublished) (1996); Chen et al., Nature 377:454-457 (1995)). In the presence of T3, the co-repressor is released from the receptor and coactivators are recruited to enhance gene expression. Many transcription factors, such as p53, WT-1, YY1, Rel, can also act as dual activators and repressors depending on the DNA template and protein co-factors with which they interact.

The Drosophila zinc finger transcription factor, Krüppel, is encoded by a gap gene and is essential for organogenesis during later stages of the development. Through in vitro protein-protein interaction studies, Sauer et al. have demonstrated that the Krüppel protein can act as a transcriptional activator at low protein concentration (monomeric form) by interacting with TFIIB. However, at higher protein concentration, Krüppel forms a dimer and directly interacts with TFIIE resulting in transcriptional repression. Several Krüppel related proteins recently have been identified in mammalian cells (Witzgall et al., Mol. Cell. Biol. 13:1933-42 (1993); Witzgall et al., Proc. Natl. Acad. Sci. 91:4514-4518 (1994); Margolin et al., Proc. Natl. Acad. Sci. 91:4509-4513 (1994)). One of them, Kid-1, was isolated from rat kidney and contains a highly conserved region of ˜75 amino acids at the N-terminus termed Krüppel-associated box (KRAB).

It has been shown that the KRAB domain can act as a potent repressor when fused to a yeast GAL4 DNA binding domain or TetR (Deuschle et al., Mol. Cell. Biol. 15:1907-1914 (1995)). Replacement of the VP16 transcriptional activation domain with the Kid-1 KRAB repression domain, converted a regulatable transactivator into a regulatable repressor. By exchanging the GAL4 DNA binding domain with the DNA binding domain of another protein, repression of a target gene (e.g., tumor proliferation gene) may be achieved in response to ligand RU486. Recently, Deuschle et al. reported that the KRAB domain isolated from Koxl zinc finger protein, which shares extensively homology with that of Kid-1, interacts with a 110 kDa adaptor protein termed SMP1 (silencing-mediating protein 1). The characteristics and mechanism of this adaptor protein have yet to be determined. Recently, a KRAB-associated protein-1 (KAP-1) was identified which binds to the KRAB domain and functions as a transcriptional co-repressor (Friedman et al., Gene and Dev. 10:2067 (1996)).

Using the newly modified GL₉₁₄VP_{C′}, regulation of neurite outgrowth in PC12 cells via RU486 controllable expression of NGF was achieved. This novel inducible system can be employed to analyze biological function in a temporal manner. For example, the role of a growth factor could be assessed at a particular stage of development and the sequential relationship of in vivo cell death and proliferation could be delineated in a manner not possible with constitutive expression of the test gene.

Tissue specific regulation of gene expression in transgenic mice utilizing this inducible system was demonstrated. RU486 inducible regulator may be used to create an inducible gene knockout (temporal and/or spatial) in transgenic mice which could circumvent an embryonic lethality resulting from use of current gene knockout techniques. Combinatorial inclusion of other inducible systems such as the tetracycline or ecdysone system with the RU486 inducible system may allow biologists one day to modulate complex biological processes which involve multiple levels of control.

The following are examples of the present invention using the mutated steroid receptors for gene therapy. It will be readily apparent to one skilled in the art that various substitutions and modifications may be made to the invention disclosed herein without departing from the scope and spirit of the invention. Thus, these examples are offered by way of illustration and are not intended to limit the invention in any manner.

The following are specific examples of preferred embodiments of the present invention. These examples demonstrate how the molecular switch mechanisms of the present invention can be used in construction of various cellular or animal models and how such molecular switch mechanisms can be used to transactivate or transrepress the regulation of gene expression. The utility of the molecular switch molecules is noted herein and is amplified upon in related applications by O'Malley et al, entitled “Modified Steroid Hormones for Gene Therapy and Methods for Their Use,” and by Vegeto, et al., entitled “Mutated Steroid Hormone Receptors, Methods for Their Use and Molecular Switch for Gene Therapy,” supra and in a related U.S. patent by Vegeto, et al., entitled “Progesterone Receptor Having C-Terminal Hormone Binding Domain Truncations,” supra. Such sections (including drawings) are hereby specifically incorporated by reference herein.

### Methods of Use

Cell Transformation

One embodiment of the present invention includes cells transformed with nucleic acid encoding for the mutated receptor. Once the cells are transformed, the cells will express the protein, polypeptide, or RNA encoded for by the nucleic acid. Cells include but are not limited to joints, lungs, muscle and skin. This is not intended to be limiting in any manner.

The nucleic acid which contains the genetic material of interest is positionally and sequentially oriented within the host or vectors such that the nucleic acid can be transcribed into RNA and, when necessary, be translated into proteins or polypeptides in the transformed cells. A variety of proteins and polypeptides can be expressed by the sequence in the nucleic acid cassette in the transformed cells.

Transformation can be done either by in vivo or ex vivo techniques. One skilled in the art will be familiar with such techniques for transformation. Transformation by ex vivo techniques includes co-transfecting the cells with DNA containing a selectable marker. This selectable marker is used to select those cells which have become transformed. Selectable markers are well known to those who are skilled in the art.

For example, one approach to gene therapy for muscle diseases is to remove myoblasts from an affected individual, genetically alter them in vitro, and reimplant them into a receptive locus. The ex vivo approach includes the steps of harvesting myoblasts cultivating the myoblasts, transducing or transfecting the myoblasts, and introducing the transfected myoblasts into the affected individual.

The myoblasts may be obtained in a variety of ways. They may be taken from the individual who is to be later injected with the myoblasts that have been transformed or they can be collected from other sources, transformed and then injected into the individual of interest.

Once the ex vivo myoblasts are collected, they may be transformed by contacting the myoblasts with media containing the nucleic acid transporter and maintaining the cultured myoblasts in the media for sufficient time and under conditions appropriate for uptake and transformation of the myoblasts. The myoblasts may then be introduced into an appropriate location by injection of cell suspensions into tissues. One skilled in the art will recognize that the cell suspension may contain: salts, buffers or nutrients to maintain viability of the cells; proteins to ensure cell stability; and factors to promote angiogenesis and growth of the implanted cells.

In an alternative method, harvested myoblasts may be grown ex vivo on a matrix consisting of plastics, fibers or gelatinous materials which may be surgically implanted in an appropriate location after transduction. This matrix may be impregnated with factors to promote angiogenesis and growth of the implanted cells. Cells can then be reimplanted.

Administration

Administration as used herein refers to the route of introduction of a vector or carrier of DNA into the body. Administration may include intravenous, intramuscular, topical, or oral methods of delivery. Administration can be directly to a target tissue or through systemic delivery.

In particular, the present invention can be used for treating disease or for administering the formulated DNA expression vectors capable of expressing any specific nucleic acid sequence. Administration can also include administering a regulatable vector discussed above. Such administration of a vector can be used to treat disease. The preferred embodiment is by direct injection to the target tissue or systemic administration.

A second critical step is the delivery of the DNA vector to the nucleus of the target cell where it can express a gene product. In the present invention this is accomplished by formulation. The formulation can consist of purified DNA vectors or DNA vectors associated with other formulation elements such as lipids, proteins, carbohydrates, synthetic organic or inorganic compounds. Examples of such formulation elements include, but are not limited to, lipids capable of forming liposomes, cationic lipids, hydrophilic polymers, polycations (e.g., protamine, polybrene, spermidine, polylysine), peptide or synthetic ligands recognizing receptors on the surface of the target cells, peptide or synthetic ligands capable of inducing endosomal lysis, peptide or synthetic ligands capable of targeting materials to the nucleus, gels, slow release matrices, soluble or insoluble particles, as well as other formulation elements not listed. This includes formulation elements for enhancing the delivery, uptake, stability, and/or expression of genetic material into cells.

The delivery and formulation of any selected vector construct will depend on the particular use for the expression vectors. In general, a specific formulation for each vector construct used will focus on vector uptake with regard to the particular targeted tissue, followed by demonstration of efficacy. Uptake studies will include uptake assays to evaluate cellular uptake of the vectors and expression of the tissue specific DNA of choice. Such assays will also determine the localization of the target DNA after uptake, and establish the requirements for maintenance of steady-state concentrations of expressed protein. Efficacy and cytotoxicity can then be tested. Toxicity will not only include cell viability but also cell function.

DNA uptake by cells associated with fluid spaces have the unique ability to take up DNA from the extracellular space after simple injection of purified DNA preparations into the fluid spaces. Expression of DNA by this method can be sustained for several months.

Incorporating DNA by formulation into particulate complexes of nanometer size that undergo endocytosis increases the range of cell types that will take up foreign genes from the extracellular space.

Formulation can also involve DNA transporters which are capable of forming a non-covalent complex with DNA and directing the transport of the DNA through the cell membrane. This may involve the sequence of steps including endocytosis and enhanced endosomal release. It is preferable that the transporter also transport the DNA through the nuclear membrane. See, e.g., the following applications all of which (including drawings) are hereby incorporated by reference herein: (1) Woo et al., U.S. Ser. No. 07/855,389, entitled “A DNA Transporter System and Method of Use” filed Mar. 20, 1992; (2) Woo et al., PCT/US93/02725, entitled “A DNA Transporter System and Method of Use”, (designating the U.S. and other countries) filed Mar. 19, 1993; and (3) continuation-in-part application by Woo et al., entitled “Nucleic Acid Transporter Systems and Methods of Use”, filed Dec. 14, 1993, assigned U.S. Ser. No. 08/167,641.

In addition, delivery can be cell specific or tissue specific by including cell or tissue specific promoters. Furthermore, mRNA stabilizing sequences (3′ UTR's) can be used to provide stabilized modified receptor molecules. Such stabilizing sequences increase the half-life of mRNAs and can be cell or tissue specific. The above is discussed in more detail in U.S. Pat. No. 5,298,422 (Schwartz et al.) and U.S. application Ser. No. 08/209,846 (Schwartz et al.), filed Mar. 9, 1994, entitled “Expression Vector Systems and Method of Use.” Both of these, the whole of which, are incorporated by reference herein, including drawings.

In a preferred method of administration involving a DNA transporter system, the DNA transporter system has a DNA binding complex with a binding molecule capable of non-covalently binding to DNA which is covalently linked to a surface ligand. The surface ligand is capable of binding to a cell surface receptor and stimulating entry into the cell by endocytosis, pinocytosis, or potocytosis. In addition, a second DNA binding complex is capable of non-covalently binding to DNA and is covalently linked to a nuclear ligand. The nuclear ligand is capable of recognizing and transporting a transporter system through a nuclear membrane. Additionally, a third DNA binding complex may be used which is also capable of non-covalently binding to DNA. The third binding molecule is covalently linked to an element that induces endosomal lysis or enhanced release of the complex from the endosome after endocytosis. The binding molecules can be spermine, spermine derivatives, histones, cationic peptides and/or polylysine. See also Szoka, C. F., Jr. et al., Bioconjug. Chem. 4:85-93 (1993); Szoka, F. C., Jr. et al., P.N.A.S., 90:893-897 (1993).

Transfer of genes directly has been very effective. Experiments show that administration by direct injection of DNA into joint tissue results in expression of the gene in the area of injection. Injection of plasmids containing the mutated receptors into the spaces of the joints results in expression of the gene for prolonged periods of time. The injected DNA appears to persist in an unintegrated extrachromosomal state. This means of transfer is the preferred embodiment.

The formulation used for delivery may also be by liposomes or cationic lipids. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acid remains biologically active. Cationic lipid formulations such as formulations incorporating DOTMA have been shown to deliver DNA expression vectors to cells yielding production of the corresponding protein. Lipid formulations may be non-toxic and biodegradable in composition. They display long circulation half-lives and recognition molecules can be readily attached to their surface for targeting to tissues. Finally, cost effective manufacture of liposome-based pharmaceuticals, either in a liquid suspension or lyophilized product, has demonstrated the viability of this technology as an acceptable drug. delivery system. See Szoka, F. C., Jr. et al., Pharm. Res., 7:824-834 (1990); Szoka, F. C., Jr. et al., Pharm. Res., 9:1235-1242 (1992).

The chosen method of delivery should result in nuclear or cytoplasmic accumulation and optimal dosing. The dosage will depend upon the disease and the route of administration but should be between 1-1000 μg/kg of body weight. This level is readily determinable by standard methods. It could be more or less depending on the optimal dosing. The duration of treatment will extend through the course of the disease symptoms, possibly continuously. The number of doses will depend upon disease, the formulation and efficacy data from clinical trials.

With respect to vectors, the pharmacological dose of a vector and the level of gene expression in the appropriate cell type includes but is not limited to sufficient protein or RNA to either: (1) increase the level of protein production; (2) decrease or stop the production of a protein; (3) inhibit the action of a protein; (4) inhibit proliferation or accumulation of specific cell types; and (5) induce proliferation or accumulation of specific cell types. As an example, if a protein is being produced which causes the accumulation of inflammatory cells within the joint, the expression of this protein can be inhibited, or the action of this protein can be interfered with, altered, or changed.

Persistent Expression Using Episomal Vectors

In each of the foregoing examples, transient expression of recombinant genes induces the desired biological response. In some diseases more persistent expression of recombinant genes is desirable. This is achieved by adding elements which enable extrachromosomal (episomal) replication of DNA to the structure of the vector. Vectors capable of episomal replication are maintained as extrachromosomal molecules and can replicate. These sequences will not be eliminated by simple degradation but will continue to be copied. Episomal vectors provide prolonged or persistent, though not necessarily stable or permanent, expression of recombinant genes in the joint. Persistent as opposed to stable expression is desirable to enable adjustments in the pharmacological dose of the recombinant gene product as the disease evolves over time.

The following samples are offered by way of illustration and are not intended to limit the invention in any way.

Formulations for Gene Delivery into Cells of the Joint

Initial experiments used DNA in formulations for gene transfer into cells of the joint. This DNA is taken up by synovial cells during the process of these cells continually resorbing and remodeling the synovial fluid by secretion and pinocytosis. Gene delivery is enhanced by packaging DNA into particles using cationic lipids, hydrophilic (cationic) polymers, or DNA vectors condensed with polycations which enhance the entry of DNA vectors into contacted cells. Formulations may further enhance entry of DNA vectors into the body of the cell by incorporating elements capable of enhancing endosomal release such as certain surface proteins from adenovirus, influenza virus hemagglutinin, synthetic GALA peptide, or bacterial toxins. Formulations may further enhance entry of DNA vectors into the cell by incorporating elements capable of binding to receptors on the surface of cells in the joint and enhancing uptake and expression.

Alternatively, particulate DNA complexed with polycations can be efficient substrates for phagocytosis by monocytes or other inflammatory cells. Furthermore, particles containing DNA vectors which are capable of extravasating into the inflamed joint can be used for gene transfer into the cells of the joint. One skilled in the art will recognize that the above formulations can also be used with other tissues as well.

### EXAMPLE 1

The homogenization buffer for hormone binding assays contained 10 mM Tris-HCl, 1.5 mM EDTA, 1 mM dithiothreitol, pH 7.4 (TESH buffer). The homogenization buffer for Western analysis of receptor contained 10 mM Tris-HCl, 2 mM EDTA, 45 mM dithiothreitol, 10% glycerol and 300 mM NaCl (TEDG+salts).

Yeast Strain

The Saccharomyces cerevisiae strain BJ3505 (MATα, pep4:HIS3, prb1-Δ1.6R, his3Δ®, lys2-801, trp1-Δ101, ura3-52, gal2, (CUP1)) was used (Yeast Genetic Stock Center, Berkeley, Calif.). All yeast transformations were carried out following the lithium acetate. transformation protocol (Ito, et al., J. Bacteriol. 153:163-168, 1983).

The PCR reactions were carried out using YEphPR-B DNA template (a YEp52AGSA-derived yeast expression plasmid containing the cDNA of hPR form-B (Misrahi, et al., Biochem. Bioph. Res. Comm. 143:740-748, 1987) inserted downstream of the yeast methallothionein-CUP1 promoter) and using three different sets of primers. In order to decrease the fidelity of the second strand polymerization reaction, buffer conditions of 1.5 mM MgCl₂, 0.1 mM dNTPs and pH 8.2 were used. About 2000 primary transformants were obtained from each region-specific library.

### EXAMPLE 2

### Yeast Mutant Screening

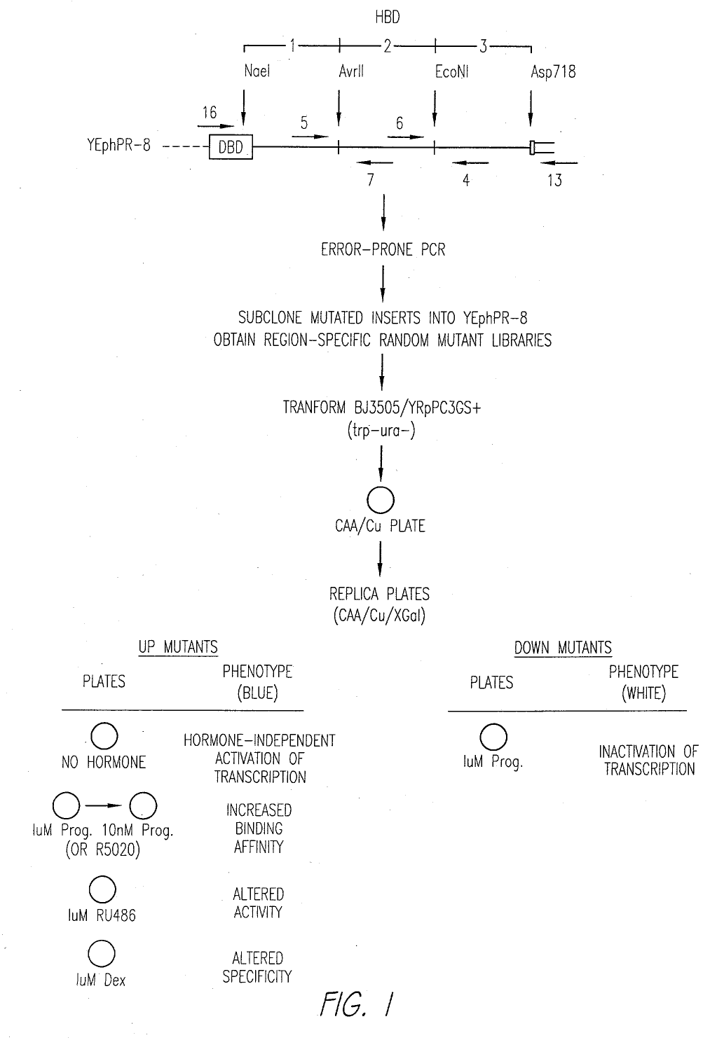

Colonies of each library of hPR molecules mutated in specific subregions were pooled, large amounts of DNA were prepared and used to transform yeast cells carrying the reporter plasmid YRpPC3GS+, which contains two GRE/PRE elements upstream of the CYC1 promoter linked to the Lac-Z gene of E. coli (Mak, et al., J. Biol. Chem. 265:20085-20086, 1989). The transformed cells were plated on 1.5% agar plates containing 2% glucose, 0.5% casamino acids (5% stock solution of casamino acids is always autoclaved before use to destroy tryptophan), 6.7 g/l yeast nitrogen base (without amino acids) and 100 μM CuSO4 (CAA/Cu plates) and grown for 2 days at 30° C. These colonies were then replica-plated on CAA/Cu plates containing 0.16 g/l of 5-bromo-4-chloro-3-indolyl-β-D-galactoside (X-Gal, an indicator of β-galactosidase activity) with or without the hormones as indicated in FIG. 1 and allowed to grow for one day at 30° C., then two days at room temperature in the dark.

### EXAMPLE 3

### Growth of Yeast Culture for in vitro Assay

Saccharomyces cerevisiae cells containing YEphPRB and the reporter plasmid were grown overnight at 30° C. in minimal media containing 2% glucose. The cells were subcultured in fresh medium and allowed to grow until early mid-log phase (O.D.₆₀₀ₙₘ=1.0). Induction of receptor was initiated by the addition of 100 μM copper sulfate to the culture. Cells were harvested by centrifugation at 1,500×g for 10 minutes and resuspended in the appropriate buffer. This and all subsequent steps of analysis of the yeast extracts were done at 4° C.

### EXAMPLE 4

### Transcription Assay

Yeast cells containing the reporter and expression plasmids were grown overnight as described above in Example 3 in the presence of 100 μM copper sulfate. When the cell density reached O.D.₆₀₀ₙₘ=1.0, hormones were added to the cultures. After a 4 hour incubation, yeast extracts were prepared and assayed for β-galactosidase activity as described previously (Miller, J. M. Miller ed., 352-355 (1972)).

Generally, reporters useful in the present invention are any which allow for appropriate measurement of transcription levels. Preferable reporter systems include reporter vectors comprised of the yeast iso-1-cytochrome C proximal promoter element fused to a structural gene, wherein said structural gene is selected from the group consisting of β-galactosidase, galactokinase and URA3. More preferably, the vector is comprised of an insertion site for a receptor response element. The vectors which include β-galactokinase as an indicator of transcriptional activity are derived from the parent vector PC2 while the vectors which include galactokinase are derived from YCpR1 vector. Preferably, the structural genes originate from E. coli.

### EXAMPLE 5

### Western Immunoblotting

Yeast cells were grown as described in Example 4 for the transcription assay. Yeast extracts for Western blot analysis were prepared by resuspending the cell pellet in TEDG+salts. The cell suspension was mixed with an equal volume of glass beads and disrupted by vortexing in a microcentrifuge tube. The homogenate was centrifuged at 12,000×g for 10 minutes. The supernatant was collected and the protein concentration was estimated using bovine serum albumin as standard. Yeast extracts were resolved on a 0.1% sodium dodecyl sulfate-7% polyacrylamide gel and transferred to Immobilon membrane as described previously (McDonnell, et al., Mol. Cell. Biol. 9:3517-3523, 1989). Solid phase radioimmunoassay was performed using a monoclonal antibody (JZB39) directed against the N-terminal domain of A and B forms of hPR.

### EXAMPLE 6

### Hormone Binding Competition Assays

Induction of PR synthesis was initiated by the addition of 100 μM CuSO₄ to the culture and incubation was continued for 6 hours. The cell pellet was resuspended in TESH buffer containing 1 μg/ml leupeptin, 10 μg/ml PMSF and 10 μg/ml pepstatin. The cell suspension was mixed with an equal volume of glass beads (0.5 mm; B. Braun Instruments) and disrupted by vortexing in a microcentrifuge tube. The homogenate was centrifuged at 12,000×g for 10 minutes and the supernatant was further centrifuged at 100,000×g for 30 minutes to obtain a cytosol fraction. Diluted yeast extracts (200 μl) containing 100 mg of total protein were incubated overnight at 4° C. with [³H]ligand in the absence (total binding) or presence (non-specific binding) of a 100-fold excess of unlabeled ligand. Bound and free steroids were separated by addition of 500 μl of dextran-coated charcoal suspension (0.5% Norit A, 0.05% dextran, 10 mM Tris HCl, pH 7.4 and 1 mM EDTA). Specific binding was determined by subtracting nonspecific from total binding. Scatchard analysis was carried out as described previously by Mak, et al., J. Biol. Chem. 264:21613-21618 (1989).

### EXAMPLE 7

### Site-Directed Mutagenesis

Mutants YEphPR-B879 and YEphPR-B891 were prepared following the procedure described by Dobson, et al., J. Biol. Chem. 264:4207-4211 (1989). CJ236 cells were infected with mpPR90 (an M13 plasmid containing hPR cDNA). The resulting uridine-containing single-stranded DNA was annealed to 20-mer oligonucleotides containing a TGA stop codon corresponding to amino acids 880 and 892, respectively.

### EXAMPLE 8

### Construction of Mammalian Expression Vectors

The mammalian expression vector phPR-B contains the SV40 enhancer sequence upstream of the human growth hormone promoter linked to the hPR-B cDNA. This vector was digested with Sall and EcoR1. The 6.1 kb fragment (containing the vector sequences and the 5′-1.5 kb of the hPR) was gel-purified and ligated to the 2.1 kb fragment of YEphPR-B891 (containing the 3′-end of the receptor) previously cut with Sal1 and EcoR1. The resulting plasmid, phPR-B891, encodes a 42 amino acid truncated version of hPR form B.

### EXAMPLE 9

### Mammalian Cell Transient Transfections and CAT-Assays

Five μg of chloramphenicol acetyltransferase (CAT) reporter plasmid, containing two copies of a PRE/GRE from the tyrosine amino transferase gene linked to the thymidine kinase promoter (PRETKCAT), were used in transient cotransfection experiments together with 5 μg of wild type or mutant receptor DNAs. Transient cotransfections and CAT-assays were performed as described by Tsai, et al., Cell 57:443-448 (1989).

### EXAMPLE 10

### Mutagenesis of the Hormone Binding Domain of hPR-B

In order to characterize amino acids within the hPR HBD which are critical for ligand binding and hormone-dependent transactivation, libraries of mutated hPR molecules were created and the mutants introduced into a reconstituted progesterone-responsive transcription system in yeast. This system allowed the screening of large numbers of mutant clones and the direct, visual identification of phenotypes.

Unique restriction sites for NaeI, AvrH and EcoNI were created in the cDNA of hPR, obtaining three cassettes of 396, 209 and 400 nucleotides (regions 1, 2 and 3, respectively). For PCR mutagenesis three sets of primers (16+7 for region 1, 5+4 for region 2 and 6+13 for region 3) were used in the polymerization reaction using YEphPR-B as DNA template. The fragments obtained after PCR were digested with the appropriate enzymes, gel-purified and ligated into the parental plasmid YEphPR-B. Ligation mixes were used to transform bacterial cells and to obtain libraries of hPR molecules randomly point-mutated in the HBD. 5 μg of DNA were used from each library to transform yeast cells carrying the reporter plasmid YRpPC3GS+ and transformants were selected for tryptophan and uracil auxotrophy on CAA plates containing 100 μM CuSO₄. These were then replicated on CAA plates containing the hormones. The screening for “up-mutations” allowed identification of receptor mutants with hormone-independent transcriptional activity, or increased affinity for the ligand (these clones should remain blue when grown with 100-fold less hormone), or with an altered response to RU38486 or a glucocorticoid analogue. In the “down-mutation” screening, receptor mutants that were transcriptionally inactive in the presence of the ligand were detected.

Because of the nature of the method used to generate the mutated DNA templates, it was necessary, firstly, to determine the quality of the libraries obtained. This was assessed by estimating the number of null-mutations generated by mutagenesis. We estimated the frequency of occurrence of transcriptionally inactive receptors (white colonies) compared to the total number of colonies. This frequency was about 7%.

The primary transformants were replica-plated onto plates containing the antiprogestin RU38486. The wild type receptor is not activated by this hormone (FIG. 1). Using this screening strategy, a single colony was identified that displayed considerable transcriptional activity in response to the antihormone. Interestingly, the same colony did not display transcriptional activity when replica-plated in the presence of progesterone. The colony was purified and the phenotype was confirmed. Eviction of the expression vector from the clone, followed by reintroduction of the unmutated receptor, demonstrated that the phenotype was indeed related to the expression vector and was not the result of a secondary mutation. In addition, the mutated plasmid called UP-1, was rescued from yeast by passage through E. coli (as described in Ward, Nucl. Acids Res. 18:5319 (1990)) and purified. This DNA was then reintroduced into yeast that contained only the reporter plasmid. As expected, the mutant phenotype was stable and related directly to the receptor expression plasmid.

### EXAMPLE 11

### Characterization of the UP-1 Mutant

The plate assays used to identify the receptor mutants are qualitative in nature. To firther characterize the properties of UP-1, the activity of the receptor mutants was compared with that of the wild type receptor in a transcription assay. In this method, yeast cells transformed with either the wild type or the mutant receptor and a progesterone responsive reporter were grown overnight in the presence of 100 μM CuSO₄. When the cells had reached an O.D.₆₀₀ₙₘ of 1.0, they were supplemented with progesterone or RU38486 and harvested by centrifugation after four hours. The β-galactosidase activity in the cell cytosol was then measured.

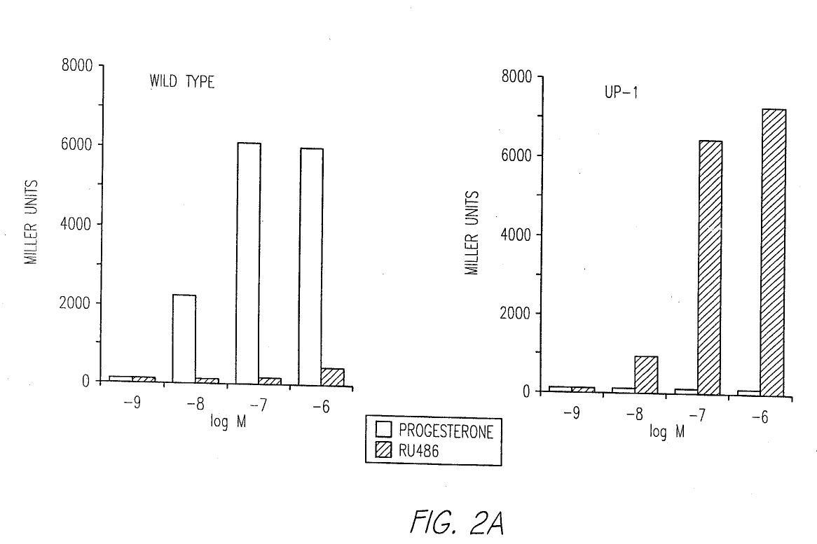

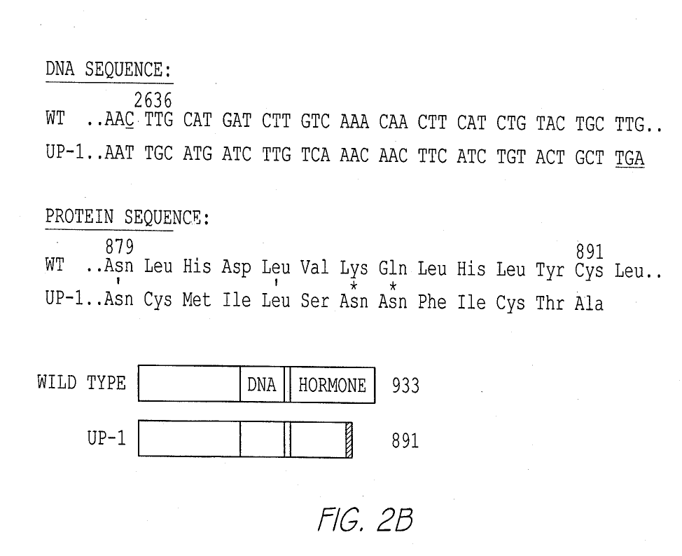

With reference to FIG. 2, panel (A), when assayed with the wild type receptor, 1 μM RU38486 is a weak inducer of transcription, whereas progesterone caused a greater than 60-fold induction of transcription at 1 μM. However, this situation was reversed when the mutant was analyzed. In this case, RU38486 was an extremely potent activator, whereas progesterone was ineffective. Interestingly, the activity achieved by the mutant in the presence of RU38486 was of the same order of magnitude as that of the wild type assayed in the presence of progesterone. This reversal in specificity clearly indicates that the mechanism by which these ligands interact with the receptor is basically different.

FIG. 2 shows the DNA and amino acid sequences of the wild type and mutant DNAs (SEQ ID NOS: 1 and 2, respectively). The cytosine at position 2636 was missing in the mutant DNA, therefore, a shifted reading frame was created and a stop codon was generated 36 nucleotides downstream of the C-2636 deletion. A schematic structure of the wild type and UP-1 receptors is also presented with a depiction of the 12 C-terminal amino acids unique to the mutant receptor. Conserved and structurally similar amino acids are marked by an apostrophe and asterisk, respectively.

DNA sequence analysis of UP-1 identified a single nucleotide deletion at base 2636 (FIG. 2B, SEQ ID NO: 2). This mutation results in a shift of the reading frame which generates a stop codon 36 nucleotides downstream. As a result, the wild type receptor is truncated by 54 authentic amino acids and 12 novel amino acids are added at the C-terminus. (Compare SEQ ID NOS: 3 and 4).

### EXAMPLE 12

### Western Analysis of the Mutant Human Progesterone Receptor

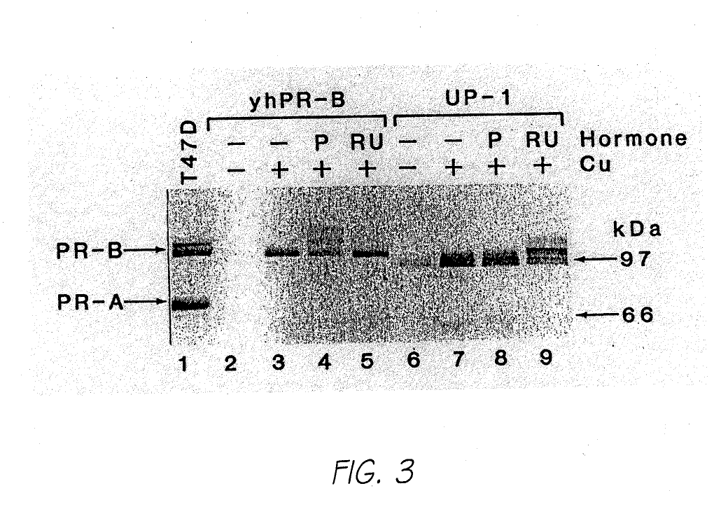

FIG. 3 shows a western analysis of mutant hPR. Yeast cells carrying the reporter plasmid and wild type (yhPR-B or mutant (UP-1) hPR were grown overnight in CAA medium with (lanes 3 to 5 and 7 to 9) or without (lanes 2 and 6) 100 μM CuSO₄. 1 μM progesterone or 1 μM RU38486 were added as indicated and cells were grown for another 4 hours. Yeast extracts were prepared as described above. 50 μg of protein extract were run on a 0.1% SDS-7% polyacrylamide gel. 50 μg of a T47D nuclear extract containing the A and B forms of hPR were also loaded (lane 1) as a positive control. The positions of molecular weight markers are indicated.

A Western immunoblot analysis of UP-1 and wild type receptors was performed in order to verify that the mutant receptor was synthesized as predicted from its DNA sequence and to eliminate the possibility that some major degradation products were responsible for the mutant phenotype. As shown in FIG. 3, the mutant receptor migrated faster in the gel, confirming the molecular weight predicted by DNA sequencing. The wild type receptor (yhPR-B) ran as a 114 kDa protein, while the mutant receptor was 5kDa smaller (compare lanes 2 and 3 with 6 and 7). The addition of 100 μM CuSO₄ to the cell cultures increased synthesis of both the wild type and mutant hPR to the same extent. No major degradation products were detected. In the presence of progesterone and RU38486, yhPR-B bands were upshifted due to hormone-induced phosphorylation of the receptor. In contrast, RU38486 induced upshifting of wild type PR to a lesser extent (lanes 4 and 5). For the UP-1 mutant this hormone-dependent upshifting was seen upon treatment with RU38486 (lanes 8 and 9). Thus, the C-terminus of PR may be responsible for the inactivity of RU38486. Consequently, removal ofthis sequence would enable RU38486 to become an agonist.

### EXAMPLE 13

### Hormone Binding Analysis

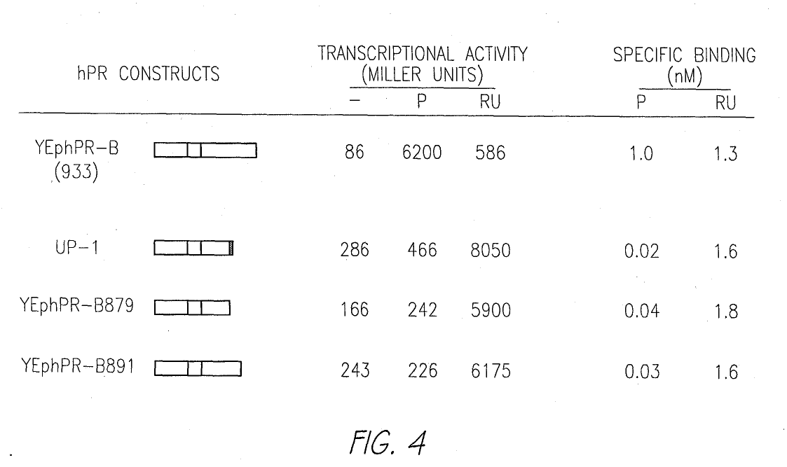

FIG. 4 shows the transcriptional activity and hormone binding analysis of wild type and mutant hPR constructs. hPR constructs are reported to the left side together with a schematic representation of the receptor molecules. Yeast cells were grown in the presence of 100 μM CuSO₄. Transcriptional analysis was done as described above. Experiments were done in triplicate and transcriptional activities were normalized with respect to protein. Hormone binding assays were performed in the presence of 20 nM [³H] progesterone or 20 nM [³H] RU38486.

A saturation binding analysis of the UP-1 mutant receptor was performed in order to determine if its affinity for RU38486 and progesterone was altered. Scatchard analysis of the binding data demonstrated that both the wild type and mutant receptors had a similar affinity for RU38486 of 4 and 3 nM, respectively. As seen in FIG. 4, the mutant receptor molecule had lost the ability to bind progesterone. Thus, the amino acid contacts for progesterone and RU38486 with hPR are different.

### Generation of Deletion Mutants of hPR-B

As shown in FIG. 2B, DNA sequencing revealed that the frameshift mutation in the UP-1 clone created a double mutation in the receptor protein. That is, a modified C-terminal amino acid sequence and a 42 amino acid truncation. In order to identify which mutation was ultimately responsible for the observed phenotype, two new receptor mutants were constructed in vitro: YEphPR-B879, containing a stop codon corresponding to amino acid 880, and YEphPR-B891, containing a stop codon at amino acid 892. Hormone binding data (see FIG. 4) demonstrated that both of these truncated receptors could bind RU38486 but not progesterone. When examined in vivo, both mutant receptors activated transcription in the presence of RU38486 to levels comparable to those of the mutant UP-1 generated in yeast. As expected, both mutants were inactive in the presence of progesterone. Thus, the observed phenotype was not due to second site mutations in the UP-1 molecule. Also, 12 additional amino acids, from 880 to 891, were not responsible for the mutant activity. In addition, it is clear the C-terminal 42 amino acids are required for progesterone to bind to the receptor while the last 54 amino acids are unnecessary for RU38486 binding. Thus, the antagonist is contacting different amino acids in the native receptor molecule and may induce a distinct receptor conformation relative to agonists.

In addition to the above deletion mutations, other deletions in the C-terminal amino acid sequence have provided binding activity with RU486 and not with progesterone. Such deletions include: (1) a 16 amino acid deletion leaving amino acids 1-917 of the progesterone receptor; and (2) a 13 amino acid deletion leaving amino acids 1-920 of the progesterone receptor. Use of the receptor binding region with TATA-CAT expression in transient transfection assays showed CAT expression with the 16 amino acid deletion, i.e., amino acids 640-917, and the 13 amino acid deletion, i.e., amino acids 640-920.

### EXAMPLE 14

### Steroid Specificity for Activation of Transcription of the UP-1 Mutant

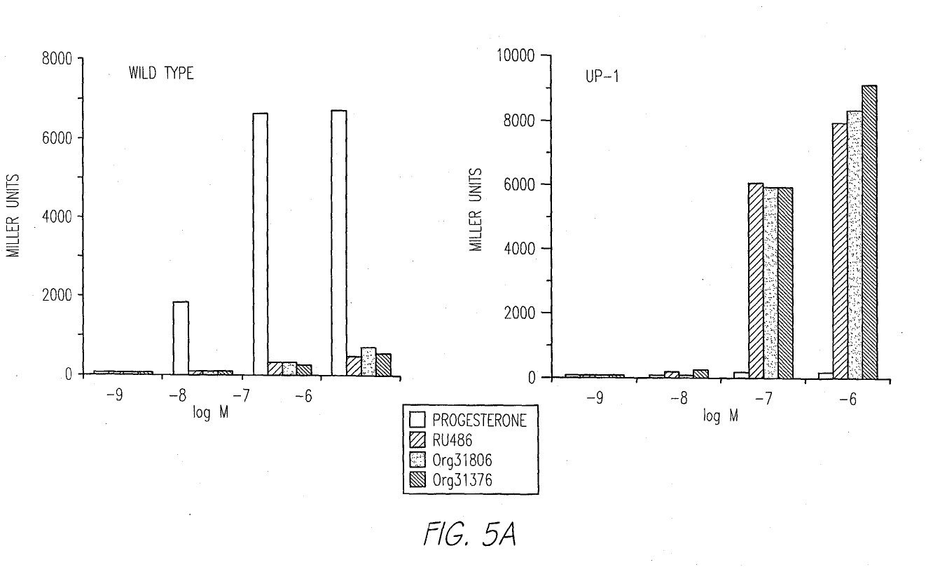

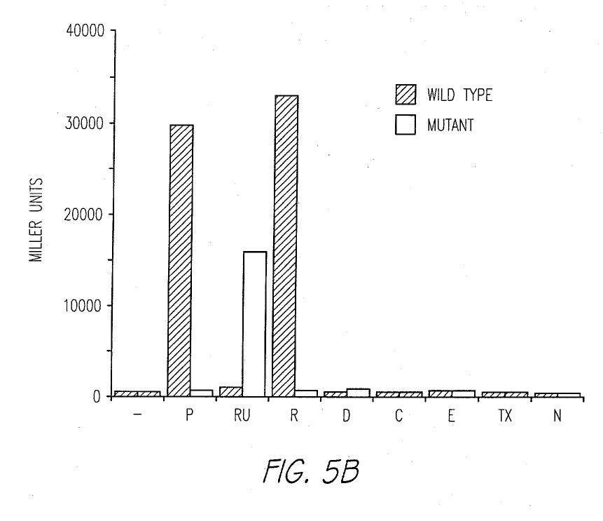

FIG. 5 shows the specificity of the transcriptional activity of the mutant hPR. In panel (A), wild type and UP-1 mutant receptor transcriptional activities were assayed in the presence of different concentrations of progesterone, RU38486, Org31806 and Org31376 as indicated.

A transcription assay was performed using two synthetic antagonists, Org31806 and Org31376, which are potent antiprogestins. As shown in FIG. 5A, the mutant receptor was activated by both of these compounds. The curve of the concentration-dependent activity was similar to that obtained with RU38486, suggesting that the affinity of these two antagonists for the mutant receptor is similar to that of RU38486. When assayed with the wild type receptor, these compounds had minimal transcriptional activity and behaved like partial agonists (3-10% of progesterone activity) only at concentrations of 1 μM, as does RU38486. Thus, the inhibitory effect of the C-terminus of hPR extends to other receptor antagonists.

In panel (B), transcriptional activities of wild type and UP-1 mutant receptors were assayed in the presence of 1 μM progesterone (P), RU38486 (RU), R5020 (R), dexamethasone (D), cortisol (C), estradiol (E), tamoxifen (TX) or nafoxidine (N) (see FIG. 5B). The synthetic agonist R5020 had no effect on the UP-1 mutant, suggesting that agonists, such as progesterone and R5020, require the C-terminus of the native receptor for binding and consequently fail to recognize the truncated UP-1 receptor. Other steroids known to enter yeast cells, such as estradiol, the antiestrogens tamoxifen and nafoxidine, dexamethasone and cortisol, might possibly activate the mutated receptor. All steroids tested were found to be inactive with either the wild type or mutant receptor. Thus, the activation of the mutant receptor is specific to antiprogestins.

### EXAMPLE 15

### Transcriptional Activity of Mutant Receptors in Mammalian Cells

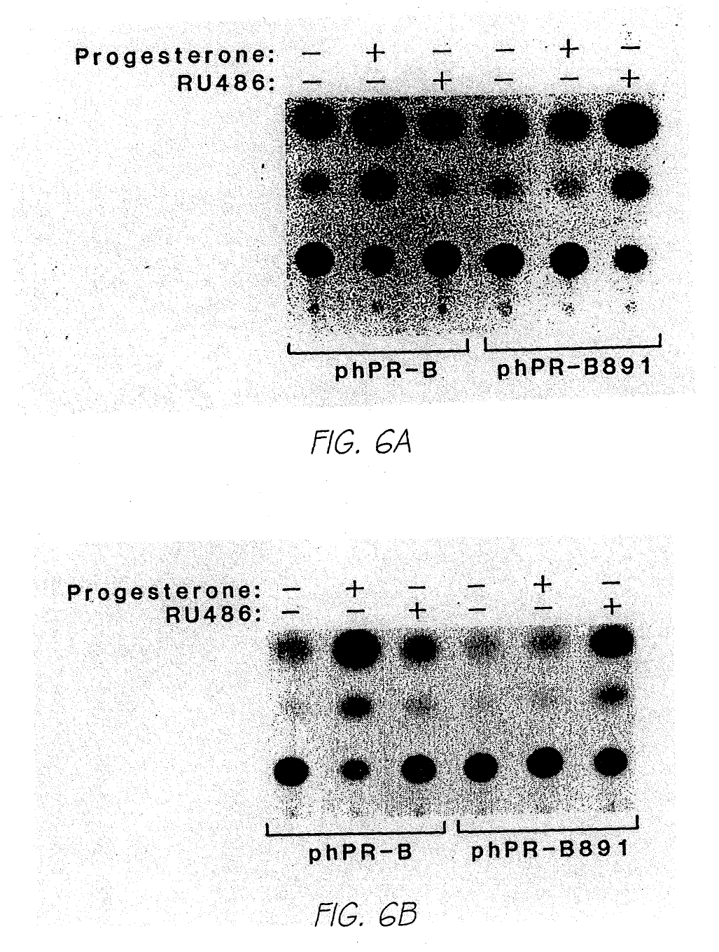

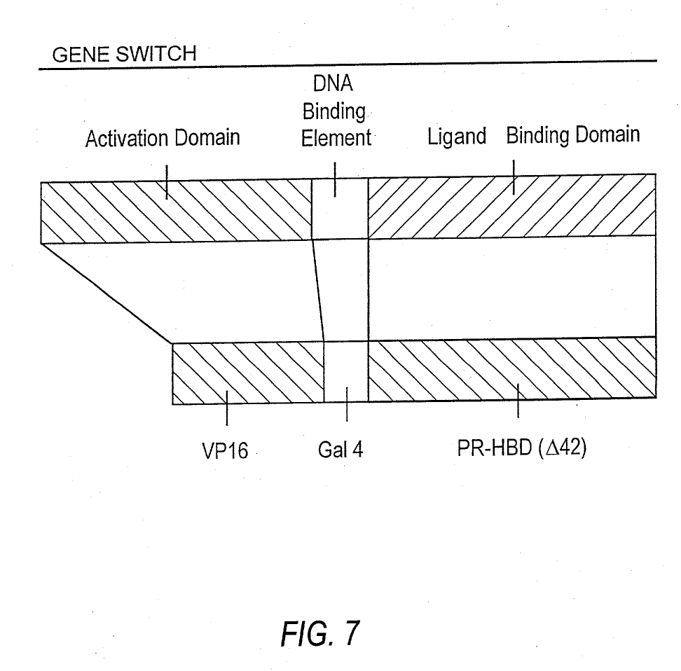

FIG. 6 shows the transient transfection of mutant hPR into mammalian cells. In panel (A), HeLa cells were transiently transfected with phPR-B and pHPR-B891 receptors together with PRETKCAT receptor plasmid using the polybrene method of transfection as described (Tsai, et al. 1989). Cells were grown with or without 100 nM progesterone or RU38486 for 48 hours prior to harvesting. CAT assays were performed as described above. In panel (B), CV-1 cells were transiently transfected as in (A).

With reference to FIG. 6, mutant receptor activity was assayed in both human endometrial HeLa cells and monkey kidney CV-1 fibroblasts. A mutant, phPR-891, was constructed by replacing the full-length PR insert of phPR-B vector with the truncated PR cDNA of YEphPR-B891. The resulting receptor mutant, phPR-B891, is a 42 amino acid truncation of hPR-B form. Mutant 891 and wild type receptors were transfected into HeLa cells together with the PRETKCAT reporter plasmid, which contains two copies of a GRE/PRE element.

As expected, wild type PR activated transcription of the CAT gene reporter in the presence of 10⁻⁷M progesterone (FIG. 6A). Although basal transcription level was high, a 3- to 4-fold induction of transcription was detected when progesterone was added to the media. In contrast, no induction occurred in the presence of RU38486. The high basal level of transcription detected in these experiments may mask or alter an RU38486 effect on wild type hPR.

On the other hand, an induction of CAT activity was observed when the 891 mutant was incubated in the presence of 10⁻⁷M RU38486 (FIG. 6A). The same concentration of progesterone had no activity.

Cell-type specific factors can influence the activity of the transactivating domains of steroid receptors. In order to evaluate this possibility, wild type and mutant receptors were transfected into CV-1 cells. Similar results were obtained, i.e., progesterone activated the wild type receptor while RU38486 activated 891 mutant receptor (FIG. 6B).

The protein synthesized from phPR-B891 plasmid was of the correct molecular weight in mammalian cells. The mutant receptor was transfected into COSM6 cells. Western analysis on cell extracts showed that the 891 mutant was synthesized, as expected, as a protein of 109 kDa, which corresponds to a protein 42 amino acids shorter than the wild type hPR. Thus, RU38486 acts as an agonist of the truncated B-receptor in a yeast reconstituted system and also in mammalian cells. The mechanism of transactivation does not require the C-terminal tail of the mutant receptor and is conserved between the three species tested.

### EXAMPLE 16

### Transgenic Animals

A molecular switch can be used in the production of transgenic animals. A variety of procedures are known for making transgenic animals, including that described in Leder and Stewart, U.S. Pat. No. 4,736,866 issued Apr. 12, 1988 and Palmiter and Bannister Annual Review of Genetics, v. 20, pp. 465-499. For example, the UP-1 molecular switch can be combined with the nucleic acid cassette containing recombinant gene to be expressed. For example, lactoferrin can be placed under the control of a basal thymidine kinase promoter into which has been placed progesterone responsive elements. This vector is introduced into the animal germ lines, along with the vector constitutively expressing the UP-1 receptor. The two vectors can also be combined into one vector. The expression of the recombinant gene in the transgenic animal is turned on or off by administering a pharmacological dose of RU 38486 to the transgenic animal. This hormone serves to specifically activate transcription of the transgene. The dose can be adjusted to regulate the level of expression. One skilled in the art will readily recognize that this protocol can be used for a variety of genes and, thus, it is useful in the regulation of temporal expression of any given gene product in transgenic animals.

### EXAMPLE 17

### Construction of Poly-Glutamine Stretch Insertion into the LBD

The poly-glutamine stretch containing multiple repeats of CAG was constructed by a method developed by S. Rusconi (Seipel et al., Nucl. Acid Res. 21:5609-5615) utilizing multimerization of DNA fragment (BsaI and BbsI digested) coding glutamine repeats leading to poly-Qₙ. Plasmid pBluscript-KS(II) was digested with Acc65I and SacI, the linearlized vector was gel purified and ligated with the annealed oligonucleotide pair R3/R4 to create plasmid pPAP. The oligonucleotide sequence for R3 (upper strand) is: 5′-GTACGTTTAAACGCGGCGCGCCGTCGACCTGCAGAAG CTTACTAGTGGTACCCCATGGAGATCTGGATCCGAATTCACGCGTTCTAGATT AATTAAGC-3′ (Seq. ID No. 5) and the sequence for R4 (lower strand) is: 5′-GGCCGCTTAATTAATCTAGAACGCGTGAATTCGGATCCAGATCTCCATGGGG TACCACTAGTAAGCTTCTGCAGGTCGACGGCGCGCCGCGTTTAAAC-3′ (Seq. ID No. 6).

The following restriction sites are incorporated into pPAP as the multiple cloning sites (from T3 to T7): PmeI, AscI, SalI, PstI, HindIII, SpeI, Acc65I, NcoI, BglII, BamHI, EcoRI, MluI, XbaI, PacI, NotI, SacI. Oligonucleotides coding for 10 glutamines were annealed and subcloned into the BglII and BamHI site of plasmid pPAP. The sequence for the upper and lower strand oligonucleotide are, 10QU 5′-GATCTCGGTCTCCAACAGCAACAGCAACAGCAACAGCAACAGGGTCTTCTG-3′ (Seq. ID No. 7) and 10QL: 5′-GATCCAGAAGACCCTGTTGCTGTTGCTGTTGCTGT TGCTGTTGGAGACCGA-3′ (Seq. ID No. 8), respectively. The insert was confirmed by restriction digestion and sequencing.

The plasmid with 10Q insert (pPAP-10Q) was digested with BsaI and BbsI (New England Biolab) overnight and precipitated. One tenth of the precipitated DNA (containing both vector and fragment) was religated to create plasmid pPAP-18Q. Each ligation step results in pAP-2(n-1)Q from the previous vector pPAP-nQ. In this way various expansion of poly-Q was achieved and resulting plasmids pPAP-34Q, pPAP-66Q and pPAP132Q were created and confirmed by sequencing. The BglII and BamHI fragment (coding for poly-Q stretch) from these plasmids were purified and cloned into BglII site of pRSV-GLVP to generate GLVP with various poly-Q insert at the N-terminus. These GLVP-nQ were reinserted into the pCEP4 vector creating pCEP4-GLVP-nQ.

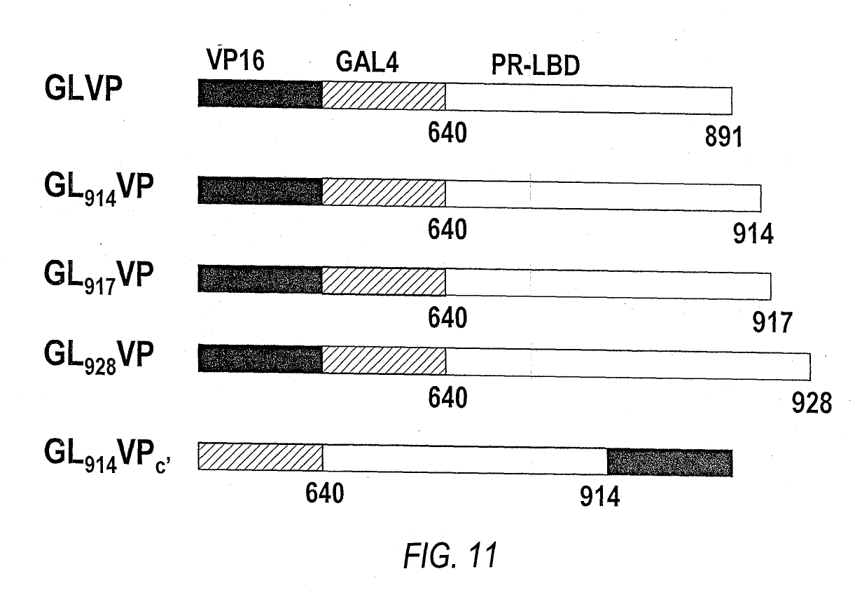

Lengthening the C-terminal ligand binding domain from 879 to 914 (FIG. 11), gradually increased RU486 induced activation of target gene expression. Importantly, these mutants responded specifically to RU486, but not to the progesterone agonist R5020. Further extension of the C-terminal LBD beyond aa 914 resulted in a decrease of GLVP response to RU486.

### EXAMPLE 18

### Chicken, Rat and Mammalian Progesterone Receptors

Chicken, rat and mammalian progesterone receptors are readily available and function by binding to the same DNA regulatory sequence. Chicken and rat progesterone receptors, however, bind a different spectrum of ligands, possessing affinities different from those interacting with human progesterone receptor. Thus, the chicken and rat progesterone receptor can be used as a transgene regulator in humans. Further, it can be used to screen for specific ligands which activate chicken or rat progesterone receptor but not endogenous human progesterone receptor. An example of a ligand is 5-alpha-pregnane-3, 20-dione (dihydroprogesterone) which binds extremely well to chicken and rat progesterone receptor but does not bind or binds very poorly to human progesterone receptor.

Although the unmodified chicken or rat progesterone receptors are already endowed with a different spectrum of ligand binding affinities from the human or other mammals and can be used in its native form, it is important to try to select additional mutated progesterone receptor to create a more efficacious receptor. The differences in chicken, rat and human progesterone receptors are due to a few amino acid differences. Thus, other mutations could be artificially introduced. These mutations would enhance the receptor differences. Screening receptor mutations for ligand efficacy produces a variety of receptors in which alterations of affinity occur. The initial screening of progesterone mutants was carried out using intermediate levels of ligands. One mutant had lost progesterone affinity entirely, but bound a synthetic ligand RU486 with nearly wild-type efficiency. RU486 is normally considered an antagonist of progesterone function, but had become an agonist when tested using this specific mutant. Because the ligand is synthetic, it does not represent a compound likely to be found in humans or animals to be treated with gene therapy. Although RU486 works as an agonist in this case, it is not ideal because of its potential side effects as an anti-glucocorticoid. Further, it also binds to the wild-type human progesterone. Thus, it has the undesirable side effect of reproductive and endocrine disjunction.

This approach is not limited to the progesterone receptor, since it is believed that all ligand activated transcription factors act through similar mechanisms. One skilled in the art recognizes that similar screening of other members of the steroid superfamily will provide a variety of molecular switches. For example, the compound 1,25-dihydroxy-Vitamin D₃ activates the Vitamin D receptor but the compound 24,25-dihydroxy-Vitamin D does not. Mutants of the Vitamin D receptor can be produced which are transcriptionally activated when bound to 24,25-dihydroxy-Vitamin D, but not by 1,25-Vitamin D₃.

One skilled in the art recognizes that the ligands are designed to be physiologically tolerated, easily cleared, non-toxic and have specific effects upon the transgene system rather than the entire organism.

### Location of Transregulatory Domains at the C-Terminal

### EXAMPLE 19

### Chimeric Fusion Protein with Various C-terminus Deletions

To construct GLVP chimeras with various C-terninal deletions of the human progesterone receptor ligand binding domain, the HindIII to BamHI fragment containing these various deletions in pRSV-hPR plasmids (Xu et al. (1996) (unpublished)) was gel purified with QIAEX II gel extraction kit (Qiagen). The purified fragments were subcloned into HindIII and BamHI sites of pRSV-GLVP (Wang et al., Proc. Natl. Acad. Sci. 91:8180-8184 (1994)) replacing the amino acid region 610 to 891 of the GLVP.

### EXAMPLE 20

### GLVP_{c′} Chimeras with VP16 Activation at the C-terminus

Two-step clonings were used to move VP16 activation to the C-terminus of the chimeric fusion protein. First, the hPR -LBD region (from amino acid 800 to various C-terminus) was amplified using 5′ primer (5′-TATGCCTTACCATGTGGC-3′ (Seq. ID No. 9)) with a different 3′ primer as a pair and digested with HindIII to SalI to prepare the fragment for ligation. For a different position of amino acid truncation, the 3′ primers incorporating the SalI site are: P3S-879: 5′-TTGGTCGACAAGATCATGCA TTATC-3′ (Seq. ID No. 10); P3S-891: 5′-TTGTCGACCCGCAGTACAGATGAAGTTG-3′ (Seq. ID No. 11) and P3S-914: 5′-TTGGTCGACCCAGCAATAACTTCAGACATC-3′ (Seq. ID No. 12). The DNA fragment containing the VP16 activation domain (amino acid 411-490) was isolated from pMSV-VP16-Δ3′-Δ58N′ with SalI and BamHI.

The digested PCR fragment and VP16 activation were ligated together into the HindIII and BamHI sites of expression vector pCEP4 (Invitrogen). The ligated vector pCEP4-PV (LBD 810-879 and VP16), -C3 (LBD 810-891 and VP16), -C2 (LBD 810-914 and VP16), respectively, now contain C-terminal fragments of hPR-LBD from the HindIII site (amino 810) to various truncations of LBD fused 3′ to VP16 activation domain with BamHI after the termination codon of VP16. The HindIII-BamHI fragment from pGL (in pAB vector) was then replaced with PV, C3, and C2 fragment, respectively, to yield PGL₈₇₉VP_{C′}, pGL₈₉₁VP_{C′}, and pGL₉1₄VP_{C′}. These chimeric fusion proteins were then subcloned into Acc65I and BamHI sites of pCEP4 expression and were named as pCEP4-GL₈₇₉VP_{C′}, pCEP4-GL₈₉₁VP_{C′}, pCEP4-GL₉₁₄VP_{C′} (FIG. 11).

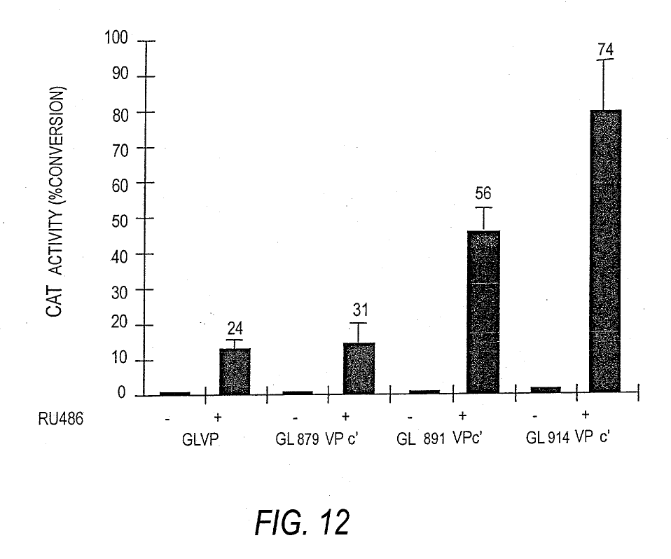

The regulator with a C-terminally located VP16 is more potent than its N-terminal counterpart (FIG. 12). In addition, extension of the C-terminal LBD from amino acid 879 to amino acid 914 further increased transactivational activity of the regulator in this C-terninally located VP16 chimera. Thus, extension of the LBD to amino acid 914 further enhances the RU486-dependent transactivation, irrespective of whether VP16 is located in the N- or C-terminus, suggesting the existence of a weak dimerization and activation function between amino acid 879 and 914 of the PR-LBD. By transferring the VP16 activation domain from the N-terminus to the C-terminus, a much more potent transactivator GL₉₁₄VP_{C′} was generated.

The modified GL₉₁₄VP_{C′} is not only more potent but also activates the reporter gene at a lower concentration of ligand as compared to GL₉₁₄VP where VP16 is located at the N-terminus. GL₉₁₄VP activity occurred at an RU486 concentration of 0.1 nM and reached a maximal level at 1 nM. In contrast, GL₉₁₄VP_{C′} increased reporter gene expression at an RU486 concentration 10 fold lower (0.01 nM) than that of GL₉₁₄VP. This newly discovered character of GL₉₁₄VP_{C′} is important for its use in inducible target gene expression, since it would allow use of a concentration which has no anti-progesterone or anti-glucocorticoid activity. This represents a significant advantage when the inducible system is applied in in vivo situations, as exemplified by transgenic mice and gene therapy.

### EXAMPLE 21

### Inducible Repressor Containing the Kid-1 KRAB Domain

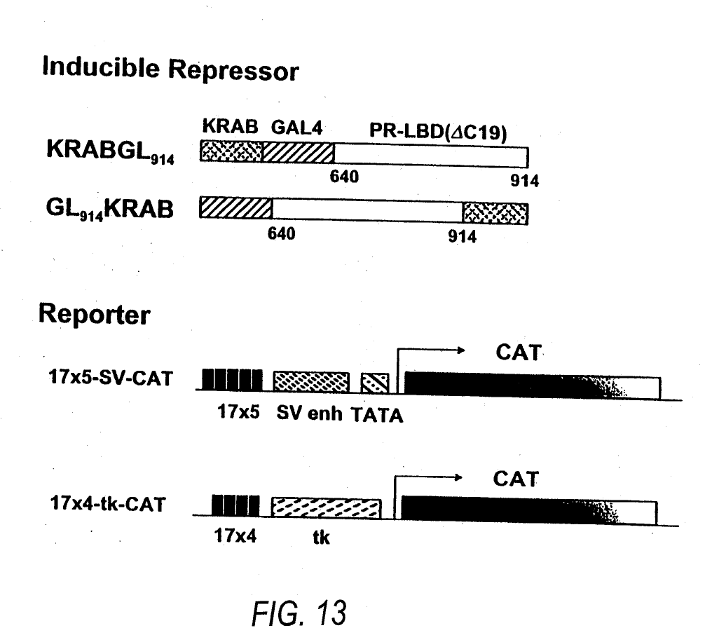

The Kid-1 gene containing the KRAB domain (aa. 1-70) was amplified with 2 sets of primers for insertion into the N- or C-terminus of GL₉₁₄, respectively. For the KRAB domain to be inserted at the N-terminus of the fusion protein, the Kid-1 cDNA was amplified with the set of primers as follows: Kid3: 5′-CGACAGATCTGGCTCCTGAG CAAAGAGAA-3′ (Seq. ID No. 13), Kid4: 5′-CCAGGGATCCTCTCCTTGCTGCAA-3′ (Seq. ID No. 14). The PCR products were digested with BglII and BamHI and subcloned into pRSV-GL₈₉₁ to create pRSV-KRABGL₈₉₁. The KpnI-SalI fragment of KRABGL₈₉₁ was then purified and subcloned into KpnI-SalI sites in pRSV-GL₉₁₄VP to create pRSV-KRABGL₉₁₄. The entire KRABGL₉₁₄ fragment (KpnI-BamHI) was then inserted into the KpnI and BamHI digested pCEP4 generating pCEP4-KRABGL₉₁₄ (FIG. 13).

For C-terminally located KRAB domain, the Kid-1 gene was amplified with the following set of primers: Kid1: 5′-TCTAGTCGACGATGGCTCCT GAGCAAAGAGAAG-3′ (Seq. ID No. 15), Kid2: 5′-CCAGGGATCCTATCCTTGCT GCAACAG (Seq. ID No. 16). The primer Kid2 also contains a termination codon (TAG) after amino acid. 70. The PCR products were digested with SalI and BamHI and purified using QIAEX II gel extraction kit (Qiagen). The HindIII and SalI fragment (317 bp) from pBS-GL₉₁₄VP_{C′}, was isolated as is the vector fragment of pCEP4-GL₉₁₄VP_{C′} digested with HindIII and BamHI. These three piece fragments were ligated to create pCEP4-GL₉₁₄KRAB.

The chimeric regulator GL₉₁₄KRAB, with the KRAB repression domain inserted in the C-terminus, strongly repressed expression (6-8 fold) of both reporters in an RU486-dependent manner. However, the N-terminally located KRAB repression domain (KRABGL₉₁₄) did not repress target gene expression in the presence of RU486 to the degree of that achieved with KRAB located in the C-terminus (GL₉₁₄KRAB).

### EXAMPLE 22

### Transient Transfection, CAT Assay, hGH Assay and Western Blot

HeLa and CV1 cells were transfected with the described amount of DNA using the polybrene mediated Ca₂PO₄ precipitation method and CAT assay was performed and quantified as described above (Wang et al., Proc. Natl. Acad. Sci. 91:8180-8184 (1994)). HepG2 cells (10⁶) were grown in DMEM with 10% fetal bovine serum and 1× Penicillin-Streptomycin-Glutamine (Gibco BRL) and transfected with polybrene mediated Ca₂PO₄ precipitation method. Aliquots of the cell culture media were taken at different time intervals and hGH production was measured using the hGH clinical assay kit (Nichols Institute) according to the manufacture's instruction. For Western blot analysis, protein extracts (20 μg) were prepared from transiently transfected HeLa cells, separated on SDS polyacrylamide gel and trans-blotted onto nylon membrane as described above. The blot was probed with anti-GAL4-DBD (aa. 1-147) monoclonal antibody (Clonetech) and developed with an ECL kit (Amersham).

These analyses confirmed that the two regulator proteins are expressed at a similar level. Together, these results suggest that through modification of the PR-LBD within the chimeric regulator we could further improve its response to a ligand by at least one order of magnitude.

### EXAMPLE 23

### Stable Cell Line Generation and Neurite Outgrowth Assay

To demonstrate the use of the inducible system in a biological situation, a regulatable expression model for nerve growth factor (NGF) was designed. NGF has been shown to stimulate neurite (axon) outgrowth of PC12 cells (from rat adrenal pheochromocytoma) when added to the cell culture media (Greene et al., Proc. Natl. Acad. Sci. 73:2424-2428 (1976)).

Rat FR cells, derived from rat fetal skin cells (American Type Culture Collection, CRL 1213) were transfected with pCEP4-GLVP₉₁₄VP_{C′} by the Ca₂PO₄ method as described previously (Wang et al., Proc. Natl. Acad. Sci. 91:8180-8184 (1994)). Cells were grown in DMEM with 10% fetal bovine serum and selected with 50 μg/ml hygromycin-B (Boehringer Mannheim). After 2-3 weeks colonies were picked and subsequently expanded. Each clone was then transiently transfected with 2 μg of the p17X4-TATA-CAT plasmid utilizing Lipofectin (GIBCO-BRL). Twenty-four hours later, the cells were treated with either RU486 (10⁻⁸M) or 80% ethanol vehicle. Cells were harvested 48 hours later and CAT activity was measured using 50 μg of cell extracts. Clones showing RU486 inducible CAT activity were subsequently transfected with the vector p17X4-TATA-rNGF(Neo).

Stable cells containing both genes were selected with hygromycin (50 μg/ml) and G418 (100 μg/ml) for 2-3 weeks and subsequently expanded. Each colony was then seeded into a 10 cm culture dish and treated with 10⁻⁸M RU486 or vehicle control (80% ethanol). After 48 hours, the conditioned media was collected and frozen. Subsequently, the conditioned media was thawed and diluted two-fold in DMEM with 10% horse serum and 5% fetal bovine serum. The diluted conditioned media was then placed on PC12 cells, with new diluted conditioned media added every two days. After 5-7 days, PC 12 cells were observed for neurite outgrowth.

When conditioned media (from C4FRNGF cells treated with RU486) was added to PC12 cells, strong neurite outgrowth from PC12 cells was observed after 48 hrs of incubation. Little if any neurite outgrowth was observed in PC12 cells incubated with the conditioned media that was collected from stable cells treated with vehicle only (85% ethanol). These results demonstrate that the inducible system can be used to control various biological phenomenon.

One skilled in the art would readily appreciate that the present invention is well adapted to carry out the objects and obtain the ends and advantages mentioned, as well as those inherent therein. The mutated steroid receptors along with the methods, procedures, treatments, molecules, specific compounds, described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

1. A molecular switch protein for regulating expression from a promoter transcriptionally linked to a nucleic acid encoding a desired gene product, said molecular switch protein comprising:
a DNA binding domain which binds the promoter; a transregulatory domain which regulates transcription from the promoter when the molecular switch protein is bound to an agonist for said molecular switch protein and to the promoter; and a mutated steroid hormone receptor superfamily protein ligand binding domain comprising a deletion of about five to about 42 carboxy terminal amino acids from a naturally occurring steroid hormone receptor superfamily protein ligand binding domain, wherein the mutation confers efficient activation of the molecular switch protein by the agonist which is an antagonist of the naturally occurring steroid hormone receptor superfamily protein.

2. The molecular switch protein of claim 1, wherein the transregulatory domain is a transactivation domain that is different from a transactivation domain that is naturally associated with the mutated steroid hormone receptor superfamily protein ligand binding domain.

3. The molecular switch protein of claim 2, wherein the transactivation domain is selected from the group consisting of VP-16, TAF-1, TAF-2, TAU-1 and TAU-2.

4. The molecular switch protein of claim 1, wherein the DNA binding domain is selected from the group consisting of glucocorticoid receptor DNA binding domain, progesterone receptor DNA binding domain and GAL-4 DNA binding domain.

5. The molecular switch protein of claim 1, wherein the DNA binding domain is selected from the group consisting of yeast DNA binding domain, a virus DNA binding domain, an insect DNA binding domain, or a non-mammalian DNA binding domain.

6. The molecular switch protein of claim 5, wherein the yeast DNA binding domain is a GAL-4 DNA binding domain.

7. The molecular switch protein of claim 1, wherein the transregulatory domain is a VP-16 transactivation domain and the DNA binding domain is GAL-4 DNA binding domain.

8. The molecular switch protein of claim 1, wherein said mutated steroid hormone receptor superfamily protein ligand binding domain is selected from the group consisting of estrogen, progesterone, glucocorticoid-α, glucocorticoid-β, mineralocorticoid, androgen, thyroid hormone, retinoic acid, retinoid X, Vitamin D, COUP-TF, ecdysone, Nurr-1 and orphan receptors.

9. The molecular switch protein of claim 8, wherein the mutated steroid hormone receptor superfamily protein ligand binding domain is a mutated progesterone receptor protein ligand binding domain.

10. The molecular switch protein of claim 9, wherein the carboxy terminal deletion comprises a deletion of about 16 to about 19 amino acids.

11. The molecular switch protein of claim 1, wherein the mutated steroid hormone receptor superfamily protein ligand binding domain binds a non-natural ligand.

12. The molecular switch protein of claim 11, wherein the mutated steroid hormone receptor superfamily protein ligand binding domain binds a non-natural ligand selected from the group consisting of 5-alpha-pregnane-3,20-dione; 11β-(4-dimethylaminophenyl)-17β-hydroxy-17α-propinyl-4,9-estradiene-3-one; 11β-(4-dimethylaminophenyl)-17α-hydroxy-17β-(3-hydroxypropyl)-13α-methyl-4,9-gonadiene-3-one; 11β-(4-acetylphenyl)-17β-hydroxy-17α-(1-propinyl)-4,9-estradiene-3-one; 11β-(4-dimethylaminophenyl)-17β-hydroxy-17α-(3-hydroxy-1(Z)-propenyl-estra-4,9-diene-3 one; (7β,11β,17β)-11-(4-dimethylaminophenyl)-7-methyl-4′,5′-dihydrospiro(ester 4,9-diene 17,2′(3′H)-furan)-3-one; (11β,14β,17α)-4′,5′-dihydro-11-(4-dimethylaminophenyl)-(spiroestra-4,9-diene-17,2′(3′H)-furan)-3-one.

13. The molecular switch protein of claim 1 or claim 9, wherein the mutated steroid hormone receptor superfamily protein ligand binding domain binds an antiprogestin.

14. The molecular switch protein of claim 13, wherein the antiprogestin is selected from the group consisting of Org31806, Org31376, and RU486.

15. The molecular switch protein of claim 14, wherein the antiprogestin is RU486.

16. A molecular switch protein for regulating expression from a promoter transcriptionally linked to a nucleic acid encoding a desired gene product, said molecular switch protein comprising:
a GAL-4 DNA binding domain which binds the promoter; a transactivation domain which regulates transcription from the promoter when the molecular switch protein is bound to an agonist for the molecular switch protein and to the promoter; and a mutated progesterone receptor protein ligand binding domain comprising a deletion of about 16 to about 42 carboxy terminal amino acids from a naturally occurring progesterone receptor protein ligand binding domain, wherein the mutation confers efficient activation of the molecular switch protein by the agonist which is an antagonist of the naturally occurring progesterone receptor protein.

17. The molecular switch protein of claim 16, wherein the mutated progesterone receptor protein ligand binding domain binds to, and is efficiently activated by, an antiprogestin.

18. The molecular switch protein of claim 17, wherein the antiprogestin is selected from the group consisting of Org31806, Org31376, and RU486.

19. The molecular switch protein of claim 18, wherein the antiprogestin is RU486.

20. A nucleic acid expression system comprising:
a first nucleic acid cassette having a first nucleic acid sequence encoding for a fusion protein; wherein the fusion protein comprises: a DNA-binding domain; a transregulatory domain; and a mutated steroid hormone receptor superfamily protein ligand binding domain comprising a deletion of about 5 to about 54 carboxy terminal amino acids from a naturally occurring steroid hormone superfamily receptor protein ligand binding domain; and a second expression cassette having a second nucleic acid sequence comprising a promoter operably linked to a nucleic acid sequence encoding a gene product, wherein the expression of the gene product is activated by binding of the fusion protein to the promoter.

21. The fusion protein of claim 20, wherein the transregulatory domain is a transactivation domain

22. The fusion protein of claim 21, wherein the transactivation domain is selected from the group consisting of VP-16, TAF-1, TA-2, TAU-1 and TAU-2.

23. The fusion protein of claim 20, wherein the DNA binding domain is selected from the group consisting of glucocorticoid receptor DNA binding domain, progesterone receptor DNA binding domain and GAL-4 DNA binding domain.

24. The fusion protein of claim 20, wherein the DNA binding domain is selected from the group consisting of a yeast DNA binding domain, a virus DNA binding domain, an insect DNA binding domain, or a non-mammalian DNA binding domain.

25. The fusion protein of claim 24, wherein the yeast DNA binding domain is a GAL-4 DNA binding domain.

26. The fusion protein of claim 20, wherein the transregulatory domain is a transactivation domain that is different from a transactivation domain that is naturally associated with the mutated steroid hormone receptor superfamily protein ligand binding domain and the DNA binding domain is GAL-4 DNA binding domain.

27. The fusion protein of claim 20, wherein said mutated steroid hormone receptor superfamily protein ligand binding domain is selected from the group consisting of estrogen, progesterone, glucocorticoid-α, glucocorticoid-β, mineralo-corticoid, androgen, thyroid hormone, retinoic acid, retinoid X, Vitamin D, COUP-TF, ecdysone, Nurr-1 and orphan receptors.

28. The fusion protein of claim 20, wherein the mutated steroid hormone receptor superfamily protein ligand binding domain binds a non-natural ligand.

29. The fusion protein of claim 28, wherein the mutated steroid hormone receptor superfamily protein ligand binding domain binds a non-natural ligand selected from the group consisting of 5-alpha-pregnane-3,20-dione; 11β-(4-dimethylaminophenyl)-17β-hydroxy-17α-propinyl-4,9-estradiene-3-one; 11β-(4-dimethylaminophenyl)-17α-hydroxy-17β-(3-hydroxypropyl)-13α-methyl-4,9-gonadiene-3-one; 11β-(4-acetylphenyl)-17β-hydroxy-17α-(1-propinyl)-4,9-estradiene-3-one; 11β-(4-dimethylaminophenyl)-17β-hydroxy-17α-(3-hydroxy-1(Z)-propenyl-estra-4,9-diene-3 one; (7β,11β,17β)-11-(4-dimethylaminophenyl)-7-methyl-4′,5′-dihydrospiro(ester-4,9-diene 17,2′(3′H)-furan)-3-one; (11β,14β,17α)-4′,5′-dihydro-11-(4-dimethylaminophenyl)-(spiroestra-4,9-diene-17,2′(3′H)-furan)-3-one.

30. The fusion protein of claim 27, wherein the mutated steroid hormone receptor superfamily protein ligand binding domain is a mutated progesterone receptor protein ligand binding domain.

31. The fusion protein of claim 30, wherein the mutated progesterone receptor superfamily protein ligand binding domain binds to, and is efficiently activated by, an antiprogestin.

32. The fusion protein of claim 31, wherein the antiprogestin is selected from the group consisting of Org31806, Org31376, and RU486.

33. The fusion protein of claim 32, wherein the antiprogestin is RU486.

34. A polynucleotide encoding a regulator protein capable of regulating transcription, the regulator protein comprising:
a DNA binding domain; a transregulatory domain; and a mutated progesterone receptor ligand binding domain having a carboxy terminal amino acid truncation of about 5 to about 54 amino acids, wherein the truncated ligand binding domain binds an anti-progestin and said binding activates the regulator protein to control transcription of an RNA encoding a desired gene product.

35. The polynucleotide encoding an inducible transcription regulator protein of claim 34, wherein the anti-progestin is RU486.

36. The polynucleotide encoding the inducible transcription regulator protein of claim 35, wherein the mutated progesterone receptor ligand binding domain has a carboxy terminal amino acid truncation of about 5 to about 42 amino acids.

37. The polynucleotide encoding the inducible transcription regulator protein of claim 36, wherein the mutated progesterone receptor ligand binding domain has a carboxy terminal amino acid truncation of about 16 to about 19 amino acids and wherein the truncation confers about a 10 fold increased sensitivity to RU486 over that of a carboxy terminal amino acid truncation of about 42 amino acids.

38. The polynucleotide encoding the inducible transcription regulator protein of claim 34, wherein the transregulatory domain is a transactivation domain.

39. A method for regulating expression of a desired gene product, comprising the steps of:
introducing into an animal cell a first nucleic acid cassette encoding a desired protein under the transcriptional control of a molecular switch promoter, and a second nucleic acid cassette encoding a molecular switch protein, wherein the molecular switch protein comprises: a DNA binding domain which binds the molecular switch promoter; a mutated steroid hormone receptor superfamily ligand binding domain which does not bind a natural ligand and binds a non-natural ligand for the steroid hormone receptor superfamily ligand binding domain, and a transregulatory domain that regulates transcription from the molecular switch promoter when the molecular switch protein is bound to the molecular switch promoter and to said non-natural ligand, wherein transcription of an RNA encoding the desired gene product is regulated by administering the non-natural ligand to the cell.

40. The method for regulating expression of the desired gene product of claim 39, wherein the first and second nucleic acid cassettes are introduced into the cell in vitro to form a transformed cell; and the transformed cell is introduced into an animal.

41. The method for regulating expression of the desired gene product of claim 40, wherein transregulation of expression of the gene product is controlled by administering the non-natural ligand to the animal in vivo.

42. The method of regulating expression of a desired gene product of claim 41, wherein the animal is a transgenic animal.

43. The method of claim 39, wherein the mutated steroid hormone superfamily receptor ligand binding domain is selected from the group consisting of estrogen, androgen, Vitamin D, COUP-TF, cis-retinoic acid, Nurr-1, thyroid hormone, mineralocorticoid, glucocorticoid-α, glucocorticoid-β, and orphan receptors.

44. The method of claim 39, wherein the molecular switch protein comprises a mutated progesterone receptor ligand binding domain together with a non-progesterone DNA binding and transactivation domains.

45. The method of claim 39, wherein the first and second nucleic acid cassettes are introduced into an animal cell in vivo.

46. The method of claim 45, wherein the animal is administered a non-natural ligand which activates the molecular switch protein to control transcription of the RNA encoding the desired gene product.

47. The method of claims 40 and 45, wherein the first and second nucleic acid cassettes are contained in separate vectors.

48. The method of claim 39, wherein the molecular switch protein comprises a non-native or modified DNA binding domain.

49. The method of claim 39, wherein the amount of gene product produced by the cell is proportional to the dose of the non-natural ligand administered.


SEQUENCE LISTING (16 sequences)

SEQ ID NO: 1 - DNA, 42 bp, Homo sapiens
aacttgcatgatcttgtcaaacaacttcatctgtactgcttg

SEQ ID NO: 2 - DNA, 42 bp, Artificial sequence
  Mutant sequence UP-1 having a stop codon generated 36 nucleotides downstream of the C-2636 deletion.
aattgcatgatcttgtcaaacaacttcatctgtactgcttga

SEQ ID NO: 3 - protein, 14 aa, Homo sapiens
NLHDLVKQLHLYCL

SEQ ID NO: 4 - protein, 13 aa, Artificial sequence
  Partial translated sequence of mutant UP-1
NCMILSNNFICTA

SEQ ID NO: 5 - DNA, 98 bp, Artificial sequence
  upper strand of oligonucleotide sequence for R3 used to create plasmid pPAP
gtacgtttaaacgcggcgcgccgtcgacctgcagaagcttactagtggtaccccatggagatctggatccgaattcacgcgttctagattaattaagc

SEQ ID NO: 6 - DNA, 98 bp, Artificial sequence
  lower strand of oligonucleotide sequence for R4 used to create plasmid pPAP
ggccgcttaattaatctagaacgcgtgaattcggatccagatctccatggggtaccactagtaagcttctgcaggtcgacggcgcgccgcgtttaaac

SEQ ID NO: 7 - DNA, 51 bp, Artificial sequence
  10QU upper strand of oligonucleotide sequence coding for 10 glutamines
gatctcggtctccaacagcaacagcaacagcaacagcaacagggtcttctg

SEQ ID NO: 8 - DNA, 51 bp, Artificial sequence
  10QL lower strand of oligonucleotide sequence coding for 10 glutamines
gatccagaagaccctgttgctgttgctgttgctgttgctgttggagaccga

SEQ ID NO: 9 - DNA, 18 bp, Artificial sequence
  5′ primer used to amplify hPR-LBD region (from amino acid 800 tovarious C-terminus)
tatgccttaccatgtggc

SEQ ID NO: 10 - DNA, 25 bp, Artificial sequence
  3′ primer incorporating the Sa1I site used to generate truncatedVP16 domains
ttggtcgacaagatcatgcattatc

SEQ ID NO: 11 - DNA, 28 bp, Artificial sequence
  3′ primer incorporating the Sa1I site used to generate truncatedVP16 domains
ttgtcgacccgcagtacagatgaagttg

SEQ ID NO: 12 - DNA, 30 bp, Artificial sequence
  3′ primer incorporating the Sa1I site used to generate truncatedVP16 domains
ttggtcgacccagcaataacttcagacatc

SEQ ID NO: 13 - DNA, 29 bp, Artificial sequence
  Kid3 primer used to amplify Kid-1 gene containing the KRAB domain
cgacagatctggctcctgagcaaagagaa

SEQ ID NO: 14 - DNA, 24 bp, Artificial sequence
  Kid4 primer used to amplify Kid-1 gene containing the KRAB domain
ccagggatcctctccttgctgcaa

SEQ ID NO: 15 - DNA, 33 bp, Artificial sequence
  Kid1 primer used to amplify Kid-1 gene for C-terminally located KRAB domain
tctagtcgacgatggctcctgagcaaagagaag

SEQ ID NO: 16 - DNA, 27 bp, Artificial sequence
  Kid2 primer used to amplify Kid-1 gene for C-terminally located KRAB domain
ccagggatcctatccttgctgcaacag